(19) United States
(12) Patent Application Publication
(10) Pub. No.: US 2017/0129911 A1
(43) Pub. Date: May 11, 2017

(54) COMPLEXES COMPRISING A PLATINUM COMPOUND AND AN IMMUNE CHECKPOINT INHIBITOR AND RELATED METHODS

(71) Applicant: Massachusetts Institute of Technology, Cambridge, MA (US)

(72) Inventors: Stephen J. Lippard, Cambridge, MA (US); Samuel Gorman Awuah, Quincy, MA (US); Mohammed Abdulfattah AlDajani, Cambridge, MA (US)

(73) Assignee: Massachusetts Institute of Technology, Cambridge, MA (US)

(21) Appl. No.: 15/348,945

(22) Filed: Nov. 10, 2016

Related U.S. Application Data
(63) Continuation of application No. 62/253,356, filed on Nov. 10, 2015.

(51) Int. Cl. C07F 15/00; A61K 31/555; A61K 9/14

(52) U.S. Cl. CPC C07F 15/0093; A61K 9/14; A61K 31/555

(57) ABSTRACT

Complexes comprising a platinum compound and an immune checkpoint inhibitor, and related methods, are generally provided. In some embodiments, the immune checkpoint inhibitor is an inhibitor for indoleamine-2,3-dioxygenase.

### RELATED APPLICATIONS

This application claims the benefit of U.S. Provisional Patent Application Ser. No. 62/253,356, filed Nov. 10, 2015, entitled “PLATINUM-IMMUNE CHECKPOINT INHIBITOR CONJUGATES: COMPOSITION AND METHODS FOR COMBINED CHEM-IMMUNOTHERAPY FOR CANCER TREATMENT,” by Lippard, et al.

### STATEMENT REGARDING FEDERALLY SPONSORED RESEARCH OR DEVELOPMENT

This invention was made with Government support under Grant No. R01 CA034992 awarded by the National Institutes of Health. The Government has certain rights in the invention.

### FIELD OF THE INVENTION

Complexes comprising a platinum compound and an immune checkpoint inhibitor (e.g., an inhibitor for indoleamine-2,3-dioxygenase), and related methods are generally provided.

### BACKGROUND OF THE INVENTION

Platinum drugs are widely used in cancer therapy. Among the platinum drugs, cisplatin, carboplatin, and oxaliplatin have FDA approval and are clinically used in the United States and elsewhere. The use of platinum(II) drugs in the treatment of malignancies has been somewhat limited because of the side effects and resistance acquired by cancer cells. An alternative to platinum(II) drug candidates is the use of substitutionally more inert platinum(IV) compounds as prodrugs derived from clinically effective platinum(II) compounds. Substitutionally inert platinum(IV) complexes are less likely to be deactivated prior to reaching their destination target in the cancer cell. The activity of platinum(IV) complexes generally involves reduction with concomitant loss of the axial ligands, affording an active platinum(II) complex that readily binds to DNA. The axial ligands which are released from the platinum(IV) complex may comprise a therapeutically active agent.

Expression of indoleamine-2,3-dioxygenase (IDO), an immunosuppressive enzyme in human tumors, leads to immune evasion and tumor tolerance. IDO is therefore a tumor immunotherapeutic target, and several IDO inhibitors are currently undergoing clinical trials. IDO inhibitors can enhance the efficacy of common cancer chemotherapeutics. Other immune checkpoint inhibitors are also known.

### SUMMARY OF THE INVENTION

In some embodiments, a complex is provided comprising Formula (I):

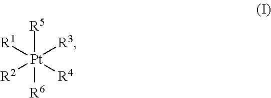

or a pharmaceutically acceptable salt thereof, wherein:

R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;

R⁵ and R⁶ can be the same or different and are —(Y)ₙR⁷, wherein each Y is the same or different and is selected from the group consisting of —O—, —NR⁸—, —C(═O)—, optionally substituted alkylene, optionally substituted heteroalkylene, optionally substituted arylene, and optionally substituted heteroarylene,

n is an integer between 1 and 10 inclusive,

each R⁷ is the same or different and is hydrogen, halo, optionally substituted alkyl, optionally substituted heteroalkyl, optionally substituted aryl, or optionally substituted heteroarylene, provided at least one R⁷ comprises an immune checkpoint inhibitor; and

each R⁸ is hydrogen, optionally substituted alkyl, or optionally substituted aryl.

In some embodiments, for a compound or Formula (I)

for a compound of Formula (I),

R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;

R⁵ and R⁶ can be the same or different and are —(Y)ₙR⁷, wherein each Y is the same or different and is selected from the group consisting of —O—, —NR⁸—, —C(═O)—, optionally substituted alkylene, optionally substituted heteroalkylene, optionally substituted arylene, and optionally substituted heteroarylene,

n is an integer between 1 and 10 inclusive,

each R⁷ is the same or different and is hydrogen, halo, optionally substituted alkyl, optionally substituted heteroalkyl, optionally substituted aryl, or optionally substituted heteroarylene, provided at least one R⁷ comprises the structure:

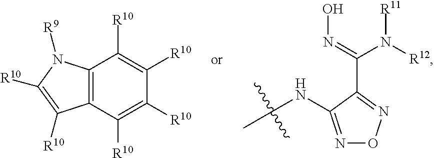

each R⁸ is hydrogen, optionally substituted alkyl, or optionally substituted aryl;

R⁹ is hydrogen, optionally substituted alkyl, or optionally substituted aryl;

each R¹⁰ is the same or different and is hydrogen, optionally substituted alkyl, optionally substituted heteroalkyl, optionally substituted aryl, or optionally substituted heteroaryl, provided at least one R¹⁰ is a bond to —(Y)ₙ—;

R¹¹ is hydrogen, optionally substituted alkyl, optionally substituted heteroalkyl, optionally substituted aryl, and optionally substituted heteroaryl; and

R¹² is optionally substituted aryl.

In some embodiments, a pharmaceutical composition is provided comprising a complex as described herein or a pharmaceutically acceptable salt, thereof and one or more pharmaceutically acceptable carriers, additives and/or diluents.

In some embodiments, a method of treating a patient in need of a therapeutic protocol is provided comprising administering to the patient a complex as described herein or a pharmaceutically acceptable salt thereof.

### BRIEF DESCRIPTION OF THE DRAWINGS

FIG. 1 shows structures of compounds, according to some embodiments.

FIG. 2A shows a plot of kynurenine inhibition by compound 2 (10 μM) in comparison to (D)-1-MT (1 mM) and an untreated control, according to some embodiments.

FIG. 2B shows immunoblotting of the pharmacological inhibition of human IDO in SKOV3 cells, according to some embodiments.

FIG. 3A shows RNAi signatures derived from the treatment of Eμ-Myc lymphoma cells with compound 2, cisplatin, and diacetate Pt(IV) at LD₈₀₋₉₀ for each compound after 72 h, according to some embodiments.

FIG. 3B shows principal component analysis plot of the RNAi signatures obtained from FIG. 3A and reference set of drugs to help elucidate the mechanism of action of compound 2, according to some embodiments.

FIG. 4A shows the estimation of T-cell population in PBMCs by measuring CD3 cell surface marker using flow cytometry, according to some embodiments.

FIG. 4B shows mixed leukocyte reaction to assess T-cell proliferation induced by compound 2 (10 μM) after 6 days of compound incubation with PBMCs and SKOV3 cells, according to some embodiments. T-cell division accounts for multiple generations (bands) measured by the intensity of CFSE dilution using flow cytometry.

FIG. 5A shows a schematic representation of PLGA-PEG nanoparticles used to encapsulate compound 2, according to some embodiments.

FIG. 5B shows time dependent stability of compound 2 in mice treated with 2-NP (8 mg/kg), according to some embodiments.

FIG. 6 shows structures of compounds, according to some embodiments.

FIG. 7A, shows Trp concentrations (μM) for untreated, hydroxyamidine-treated, and compound 2-1-treated media, according to some embodiments.

FIG. 7B shows Kyn concentrations for untreated, hydroxyamidine-treated, and compound 2-1-treated media, according to some embodiments.

FIG. 7C shows normalized concentrations for untreated, hydroxyamidine-treated, and compound 2-1-treated media, according to some embodiments.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Complexes and related methods useful for treating subjects having cancer or at risk of developing cancer are generally provided. The complex generally comprises a platinum compound and an immune checkpoint inhibitor (e.g., an inhibitor for indoleamine-2,3-dioxygenase (IDO)). The subject matter of the present invention involves, in some cases, interrelated products, alternative solutions to a particular problem, and/or a plurality of different uses of one or more systems and/or articles.

In some embodiments, the complex comprises a platinum compound and an immune checkpoint inhibitor (e.g., an inhibitor for IDO). The inhibitor may be covalently bound to the platinum metal center. Following administration, a therapeutically active platinum agent may form as well as the inhibitor may be release from the compound. The methods and complexes of the present invention which comprises a therapeutically active platinum agent and an inhibitor may be beneficial over use of either therapies used alone. For example, the treatment of a cancer with a complex as described herein may be more effective than treatment of the cancer with a therapeutically active platinum agent alone or an inhibitor alone, as described in more detail below.

It should be understood, that while much of the discussion herein focuses on inhibitors for IDO, this is by no means limiting and those of ordinary skill in the art will be able to apply the teaches herein to other immune checkpoint inhibitors. Non-limiting examples of immune checkpoint inhibitors include those that target cytotoxic T-lymphocyte-associated antigen 4 (CTLA-4) (e.g., ipilumumab and other anti-CTLA4) and programmed cell death-1 (PD-1) and PD-1 ligand (PD-L1) receptors (e.g., nivolumab, pembrolizumab, atezolizumab).

In some embodiments, a complex comprises Formula (I):

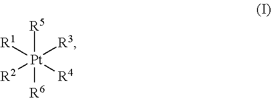

or a pharmaceutically acceptable salt thereof, wherein:

R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;

R⁵ and R⁶ can be the same or different and are —(Y)ₙR⁷,

each Y is the same or different and is selected from the group consisting of —O—, —NR⁸—, —C(═O)—, optionally substituted alkylene, optionally substituted heteroalkylene, optionally substituted arylene, and optionally substituted heteroarylene,

n is an integer between 1 and 10 inclusive,

each R⁷ is the same or different and is hydrogen, halo, optionally substituted alkyl, optionally substituted heteroalkyl, optionally substituted aryl, or optionally substituted heteroarylene, provided at least one R⁷ comprises an immune checkpoint inhibitor, and

each R⁸ is hydrogen, optionally substituted alkyl, or optionally substituted aryl. In some embodiments, the immune checkpoint inhibitor is an inhibitor for IDO.

In some embodiments, a complex comprises Formula (I):

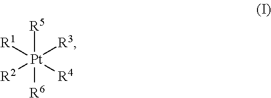

or a pharmaceutically acceptable salt thereof, wherein:

R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;

R⁵ and R⁶ can be the same or different and are —(Y)ₙR⁷,

each Y is the same or different and is selected from the group consisting of —O—, —NR⁸—, —C(═O)—, optionally substituted alkylene, optionally substituted heteroalkylene, optionally substituted arylene, and optionally substituted heteroarylene,

n is an integer between 1 and 10 inclusive,

each R⁷ is the same or different and is hydrogen, halo, optionally substituted alkyl, optionally substituted heteroalkyl, optionally substituted aryl, or optionally substituted heteroarylene, provided at least one R⁷ comprises the structure:

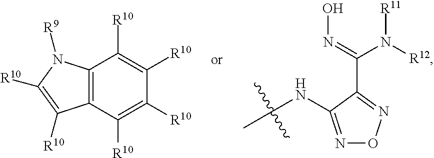

each R⁸ is hydrogen, optionally substituted alkyl, or optionally substituted aryl;

R⁹ is hydrogen, optionally substituted alkyl, or optionally substituted aryl;

each R¹⁰ is the same or different and is hydrogen, optionally substituted alkyl, optionally substituted heteroalkyl, optionally substituted aryl, or optionally substituted heteroaryl, provided at least one R¹⁰ is a bond to —(Y)ₙ—;

R¹¹ is hydrogen, optionally substituted alkyl, optionally substituted heteroalkyl, optionally substituted aryl, or optionally substituted heteroaryl; and

R¹² is optionally substituted aryl.

In some embodiments, the complex of Formula (I) is a therapeutically active salt. In some embodiment the complex having Formula (I) has the formula:

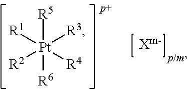

wherein X is a counterion; p is 1 or 2; and m is 1, 2, or 3. In some embodiments, p and m are each 1. In some embodiments, p and m are each 2. In some embodiments, the counterion X may be a weak or non-nucleophilic stabilizing ion. In some cases, the counterion is a negatively-charged and/or non-coordinating ion. Examples of counterions include halides, such as chloride.

In some embodiments, for a complex of Formula (I), at least one or R⁵ and R⁶ comprises the structure:

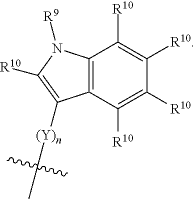

In some embodiments, for a complex of Formula (I), each of R⁵ and R⁶ comprises the structure:

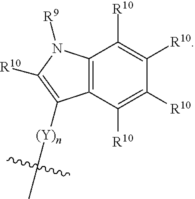

In some embodiments, for a complex of Formula (I), R⁵ comprises the structure:

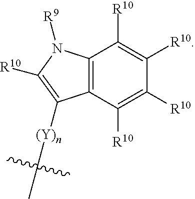

In some embodiments, R⁹ is optionally substituted alkyl. In some embodiments, R⁹ is unsubstituted alkyl. In some embodiments, R⁹ is methyl, ethyl, isopropyl, n-propyl, n-butyl, iso-butyl, or t-butyl. In some embodiments, R⁹ is methyl. In some embodiments, R⁹ is hydrogen. In some embodiments, R⁹ is optionally substituted aryl. In some embodiments, R⁹ is phenyl. In some embodiments, each R¹⁰ is hydrogen. In some embodiments, each R¹⁰ is hydrogen or optionally substituted alkyl, provided at least one R¹⁰ is a bond to —(Y)ₙ—. In some embodiments, each R¹⁰ is hydrogen or unsubstituted alkyl, provided at least one R¹⁰ is a bond to —(Y)ₙ—. In some embodiments, the group comprises the structure:

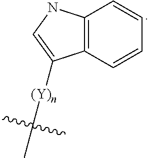

In some embodiments, each of or at least one of R⁵ or R⁶ comprises the structure:

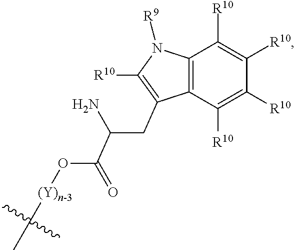

wherein R⁹, R¹⁰, Y, and n are as described herein. In some embodiments, each of or at least one of R⁵ or R⁶ comprises the structure:

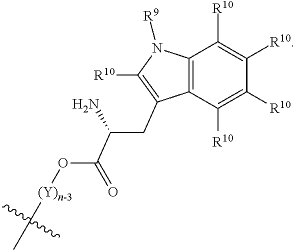

In some embodiments, each of or at least one of R⁵ or R⁶ comprises the structure:

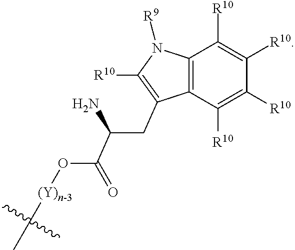

In some embodiments, for a complex of Formula (I), at least one or R⁵ and R⁶ comprises the structure:

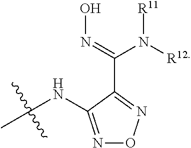

In some embodiments, for a complex of Formula (I), each of R⁵ and R⁶ comprises the structure:

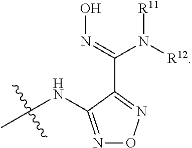

In some embodiments, for a complex of Formula (I), R⁵ comprises the structure:

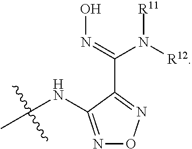

In some embodiments, R¹¹ is hydrogen. In some embodiment, R¹¹ is optionally substituted alkyl. In some embodiments, R¹¹ is unsubstituted alkyl. In some embodiments, R¹¹ is optionally substituted aryl. In some embodiments, R¹² is optionally substituted phenyl. In some embodiments, R¹² is phenyl substituted with halo. In some embodiments, R¹² is phenyl substituted with chloro. In some embodiments, the group comprises the structure:

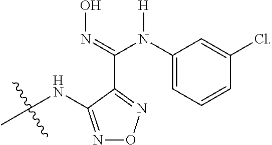 [SMILES: *Nc1nonc1/C(=N/O)Nc1cccc(Cl)c1]

The group —(Y)ₙ— may be any suitable linking group. In some embodiments, the linking group may be selected to allow for release of R⁵ and/or R⁶ under the right conditions. Non-limiting examples of functional groups which can be used in the linking group include ester, amide, amine, and anhydride moieties. In some embodiments, the group is bound to the platinum metal center via a ester moiety. In such embodiments the linking group may be hydrolyzed, in some cases, by intracellular esterases and R⁵ and/or R⁶ may be released. In some embodiments, —(Y)ₙ— comprises the structure —O—C(═O)—(Y)₍ₙ₋₂₎—. In some embodiments, —(Y)ₙ— comprises the structure —O—C(═O)-alkylene-(Y)₍ₙ₋₃₎. In some embodiments, —(Y)ₙ— is —O—C(═O)-alkylene-C(═O)—NH- alkylene-O—C(═O)—CHNH₂-alkylene-, —O—C(═O)—CHNH₂-alkylene-, or —O—C(═O)-alkylene-(C═O)—.

In some embodiments, for a complex of Formula (I), n is 1 to 10, or 1 to 8, or 1 to 5, or 1, or 2, or 3, or 4, or 5, or 6, or 7, or 8, or 9, or 10.

In some embodiments, one of R⁵ or R⁶ comprises the structure:

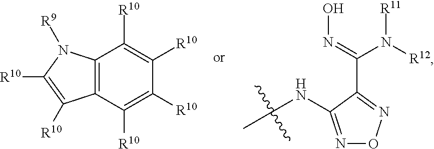

and the other one of R⁵ or R⁶ comprises the structure —(Y)ₙ—(C₁₀₋₂₄ alkyl) or —O—C(═O)—NH—(C₁₀₋₂₄ alkyl). Without wishing to be bound by theory, the presence of a hydrophobic chain, for example, a long chain alkyl group, may facilitate non-covalent binding to human serum albumin, protecting the Pt(IV) center from premature reduction in blood. In some embodiments, R⁵ or R⁶ comprises the structure —(Y)ₙ—(C₁₆ alkyl) or —O—C(═O)—NH—(C₁₆ alkyl).

In some embodiments, for a complex of Formula (I), each of R¹ and R² is NH₃. In some embodiments, for a complex of Formula (I), each of R³ and R⁴ is chloro. In some embodiments, for a compound of complex (I), each of R¹ and R² is NH₃ and each of R³ and R⁴ is chloro. In some embodiments R¹ and R² are joined together and are —O(C═O)(C═O)O—. In some embodiments, R³ and R⁴ are joined together and are diaminocyclohexane. In some embodiments R¹ and R² are joined together and are —O(C═O)(C═O)O—, and R³ and R⁴ are joined together and are diaminocyclohexane. Additional details regarding the selection of R¹-R⁴ are described herein.

As will be known to those of ordinary skill in the art, a platinum(IV) compound may be more likely to undergo a redox change following uptake into a cell. That is, the reducing environment of a cell may enhance the ability of the agent to be reduced from a platinum(IV) center to a platinum(II) center. Reduction of the metal center and release of ligands (e.g., R⁵ and R⁶ in Formula (I)) may form a therapeutically active platinum(II) agent. In some embodiments, a reducing environment within a cell may facilitate or enhance a redox change at the platinum center, precipitating release of the ligands and formation of the therapeutically active platinum(II) agent. By this mechanism, for certain subject coordination complexes, release of a covalently attached inhibitor (e.g., for IDO) can occur (or be more likely to occur) in the cell upon reduction of the platinum(IV) center to which the inhibitor is attached. By this means, the therapeutically active platinum(II) agent (or a precursor thereof) and the inhibitor (e.g., for IDO) may be generated in the same cell simultaneously. It may be the case that the therapeutically active platinum(II) agent and the inhibitor (e.g., for IDO) may act synergistically or independently.

In some embodiments, upon release of R⁵ and R⁶, a therapeutically active platinum(II) agent forms or a precursor to the therapeutically active platinum(II) agent forms. In some embodiments, dissociation of R⁵ and R⁶ from the platinum center of a complex of Formula (I) forms a compound having Formula (II),

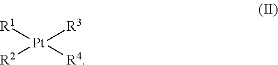

In some embodiments, the compound having Formula (II) is a therapeutically active platinum(II) agent. In some embodiments, the compound having Formula (II) comprises cisplatin, carboplatin, or oxaliplatin. In some embodiments, the compound having Formula (II) comprises cisplatin. In some embodiments, the compound having Formula (II) comprises oxaliplatin.

In other embodiments, a precursor to a therapeutically active platinum(II) agent may form upon release of R⁵ and/or R⁶, wherein the precursor may be chemically alter, transformed, and/or activated to form the therapeutically active platinum(II) agent. For example, the precursor to a therapeutically active platinum(II) agent may comprise a functional group which may undergo a chemical reaction (e.g., in situ, upon exposure to a cellular environment) to form a therapeutically active platinum(II) agent. As a specific non-limiting example, the second generation precursor may comprise a carboxylic group, which may undergo transformation in situ to form an alcohol or ester, which may be a therapeutically active composition. As another example, the replacement of a ligand on a second generation precursor of a therapeutically active platinum agent may form a therapeutically active platinum agent.

In some embodiments, one or more of R¹-R⁴ may dissociate and one or more new ligands may associate to form the therapeutically active platinum(II) agent. As a specific non-limiting example, in some embodiments, upon exposure to a cellular environment, R³, R⁴, R⁵, and R⁶ may dissociate from the platinum center, and at least two new ligands may associate with the platinum center (e.g., R¹³ and R¹⁴, as shown in Equation 1) to form a therapeutically active platinum(II) agent (e.g., [Pt(R¹)(R²)(R¹³)(R¹⁴)]), wherein at least one of R⁵ and R⁶ comprises the inhibitor (e.g., for IDO).

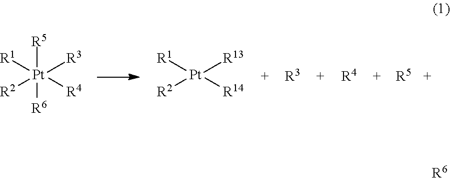

R¹³ and R¹⁴ may be the same or different and may be any suitable ligand as will be known to those of ordinary skill in the art, and are generally ligands or groups present in the environment surrounding the compound during dissociation of R³, R⁴, R⁵ and/or R⁶ (e.g., present in situ and/or in a cellular environment) and are capable of binding to platinum (e.g., water). It should be understood, that in some cases, less than all of R³, R⁴, R⁵, and R⁶ may dissociate from the platinum center and less than two ligands may associate with the platinum center. For example, R³, R⁵, and R⁶ may dissociate from the platinum center and R¹⁴ may associate, thereby forming a compound having the formula [Pt(R¹)(R²)(R³)(R¹⁴)]. Those of ordinary skill in the art will be able to select appropriate combinations of ligands to form the desired therapeutically active complex.

In some embodiments, those of R¹-R⁴ which are not essentially ligand for the formation of a therapeutically active platinum(II) agent may be a leaving group, a non-interfering ligand, and/or a non-interfering group. As used herein, the term “non-interfering group,” or “non-interfering ligand” refers to any group or ligand which does not significantly affect or alter the properties of the compound and, in some cases, does not affect or does not significantly affect a cellular pathway of a cancer cell.

In some embodiments, the ligands of the composition may be selected such that upon reduction of the metal center, one or more ligands may be released and selected therapeutically active platinum(II) agent or a precursor to a therapeutically active platinum(II) agent is formed. For example, R¹, R², R³, and R⁴ may be selected such that, upon reduction of the platinum metal center and release of R⁵ and R⁶ (as described herein), a selected therapeutically active platinum(II) agent is formed. As another example, R¹, R², may be selected such that, upon reduction of the platinum metal center, release of R³, R⁴, R⁵ and R⁶, and association of R¹³ and R¹⁴ (as described herein), a selected therapeutically active platinum(II) agent is formed. The therapeutically active platinum(II) agent may be any known platinum(II) therapeutically active platinum(II) agent. Non-limiting examples of therapeutically active platinum(II) agents include cisplatin ([cis-Pt(NH₃)₂Cl₂]), carboplatin ([cis-Pt(NH₃)₂(1,1-(OCO)C₄H₆)]), oxaliplatin, [cis-Pt(NH₃)₂(trans-1,2-(OCO)₂C₆H₁₀)], [cis-Pt(DACH)Cl₂] (where DACH is diaminocyclohexane), nedaplatin ([cis-Pt(NH₃)₂OCH₂CHO₂], stratoplatin, paraplatin, platinol, cycloplatam, dexormaplatin, enloplatin, iproplatin, lobaplatin, ormaplatin, spiroplatin, zeniplatin, etc., as will be known to those of ordinary skill in the art.

In some embodiments, the ligands associated with the platinum center in the therapeutically active platinum compound (e.g., R¹-R⁴) may include functional groups capable of interaction with a metal center, e.g., heteroatoms such as nitrogen, oxygen, sulfur, and phosphorus. Non-limiting examples of compounds which the ligands may comprise include amines (primary, secondary, and tertiary), aromatic amines, amino groups, amido groups, nitro groups, nitroso groups, amino alcohols, nitriles, imino groups, isonitriles, cyanates, isocynates, phosphates, phosphonates, phosphites, (substituted) phosphines, phosphine oxides, phosphorothioates, phosphoramidates, phosphonamidites, hydroxyls, carbonyls (e.g., carboxyl, ester and formyl groups), aldehydes, ketones, ethers, carbamoyl groups, thiols, sulfides, thiocarbonyls (e.g., thiolcarboxyl, thiolester and thiolformyl groups), thioethers, mercaptans, sulfonic acids, sulfoxides, sulfates, sulfonates, sulfones, sulfonamides, sulfamoyls, and sulfinyls. In other cases, at least some of the ligands (e.g., R¹-R⁴) may be aryl group, alkenyl group, alkynyl group or other moiety which may bind the metal atom in either a sigma- or pi-coordinated fashion. In some cases, R¹ and R² may be labile ligands and R³ and R⁴ may be non-labile ligands covalently bonded to the platinum metal center.

In some embodiments, any two or three of R¹, R², R³, and R⁴ can be joined together to form a bidentate ligand or tridentate ligand. A bidentate ligand when bound to a metal center, forms a metallocycle structure with the metal center. Bidentate ligands suitable for use in the present invention include species which have at least two sites capable of binding to a metal center. For example, the bidentate ligand may comprise at least two heteroatoms that coordinate the metal center, or a heteroatom and an anionic carbon atom that coordinate the metal center. Examples of bidentate ligands suitable for use in the invention include, but are not limited to, alkyl and aryl derivatives of moieties such as amines, phosphines, phosphites, phosphates, imines, oximes, ethers, hybrids thereof, substituted derivatives thereof, aryl groups (e.g., bis-aryl, heteroaryl-substituted aryl), heteroaryl groups, and the like. Specific examples of bidentate ligands include ethylene diamine, 2,2′-bipyridine, acetylacetonate, oxalate, and the like. Non-limiting examples of bidentate ligands include diimines, pyridylimines, diamines, imineamines, iminethioether, iminephosphines, bisoxazoline, bisphosphineimines, diphosphines, phosphineamine, salen and other alkoxy imine ligands, amidoamines, imidothioether fragments and alkoxyamide fragments, and combinations of the above ligands.

In some embodiments, a complex may comprise a tridentate ligand, which includes species which have at least three sites capable of binding to a metal center. For example, the tridentate ligand may comprise at least three heteroatoms that coordinate the metal center, or a combination of heteroatom(s) and anionic carbon atom(s) that coordinate the metal center. Non-limiting examples of tridentate ligands include 2,5-diiminopyridyl ligands, tripyridyl moieties, triimidazoyl moieties, tris pyrazoyl moieties, and combinations of the above ligands.

Pt(II) and Pt(IV) complexes described herein may be synthesized according to methods known in the art, including various methods described herein. For example, the method may comprise reaction of cisplatin with one or more ligand sources. In some cases, a Pt(IV) complex, wherein R⁵ and R⁶ are —OH, can be obtained by reaction of the parent Pt(II) species with, for example, hydrogen peroxide at temperatures ranging between about 25 and about 60° C. in an appropriate solvent, such as water or N,N-dimethylformamide. The desired Pt(IV) complex may synthesized comprising selected R⁵ and R⁶ groups according to method known in the art, for example, by functionalization of the —OH groups (e.g., by reaction with an anhydride, an isocyanate, a pyrocarbonate, an acid chloride, etc.).

In some embodiments, a platinum complex may comprise one or more leaving groups. As used herein, a “leaving group” is given its ordinary meaning in the art and refers to an atom or a group capable of being displaced by a nucleophile. Examples of suitable leaving groups include, but are not limited to, halides (such as chloride, bromide, and iodide), pyridine, alkanesulfonyloxy, arenesulfonyloxy, alkyl-carbonyloxy (e.g., acetoxy, carboxylate), arylcarbonyloxy, mesyloxy, tosyloxy, trifluoromethane-sulfonyloxy, aryloxy, methoxy, N,O-dimethylhydroxylamino, pixyl, oxalato, malonato, and the like. A leaving group may also be a bidentate, tridentate, or other multidentate ligand. In some embodiments, the leaving group is a halide or carboxylate. In some embodiments, the leaving group is chloride.

Some embodiments comprise complexes having two leaving groups positioned in a cis configuration, i.e., the compound may be a cis isomer. However, it should be understood that complexes may also have two leaving groups positioned in a trans configuration, i.e., the compound may be a trans isomer. Those of ordinary skill in the art would understand the meaning of these terms.

It should be understood that homologs, analogs, derivatives, enantiomers, diastereomers, tautomers, cis- and trans-isomers, and functionally equivalent compositions of complex described herein may also be contemplated. “Functionally equivalent” generally refers to a composition capable of treatment of patients having cancer, or of patients susceptible to cancers. It will be understood that the skilled artisan will be able to manipulate the conditions in a manner to prepare such homologs, analogs, derivatives, enantiomers, diastereomers, tautomers, cis- and trans-isomers, and functionally equivalent compositions. Homologs, analogs, derivatives, enantiomers, diastereomers, tautomers, cis- and trans-isomers, and functionally equivalent compositions which are about as effective or more effective than the parent compound are also intended for use in the method of the invention. Such compositions may also be screened by the assays described herein for increased potency and specificity towards a cancer, preferably with limited side effects. Synthesis of such complexes may be accomplished through typical chemical modification methods such as those routinely practiced in the art. Another aspect of the present invention provides any of the above-mentioned complexes as being useful for the treatment of cancer.

In some embodiments, a complex is provided having the structure:

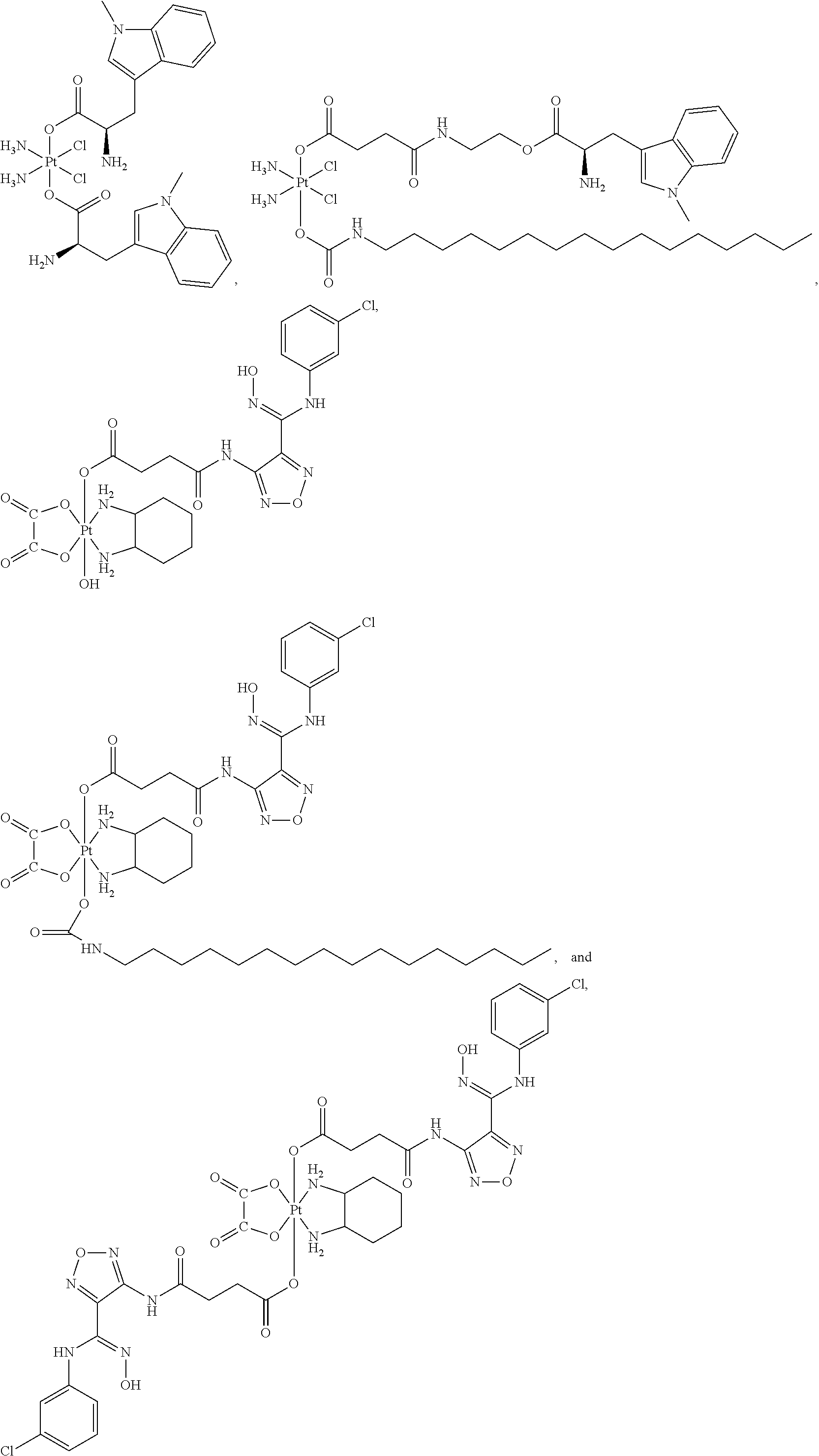

or a pharmaceutically acceptable salt thereof.

In some embodiments, a platinum complex as described herein may be associated with and/or contained within a particle. In some embodiments, a particle is provided comprising a polymeric material and a platinum complex as described herein. In some embodiments, a particle is provided comprising a polymeric material and a complex as described herein encapsulated or dispersed in the polymeric material, wherein the complex is not associated with the polymeric material via a covalent bond. In some cases, a composition is provided comprising a plurality of particles.

In some cases, a particle may be a nanoparticle, i.e., the particle has a characteristic dimension of less than about 1 micrometer, where the characteristic dimension of a particle is the diameter of a perfect sphere having the same volume as the particle. A plurality of particles, in some embodiments, may be characterized by an average diameter (e.g., the average diameter for the plurality of particles). In some embodiments, a diameter of the particles may have a Gaussian-type distribution. In some cases, the plurality of particles may have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm in some cases. In some embodiments, the particles may have an average diameter of at least about 5 nm, at least about 10 nm, at least about 30 nm, at least about 50 nm, at least about 100 nm, at least about 150 nm, or greater. In some cases, the plurality of the particles have an average diameter of about 10 nm, about 25 nm, about 50 nm, about 100 nm, about 150 nm, about 200 nm, about 250 nm, about 300 nm, about 500 nm, or the like. In some cases, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. The particle may be of any suitable size or shape. Non-limiting examples of suitable shapes include spheres, cubes, ellipsoids, tubes, sheets, and the like. Generally, the particle is spherical.

Without wishing to be bound by theory, the size of a particle may alter the delivery (e.g., loss of payload, drug efflux, aggregations, delivery to desired location, etc.) of a platinum complex from the particles. In some cases, larger particles may lose their payload more quickly than smaller particles and/or a compound efflux may be more rapid from smaller particles than larger particles. Smaller particles, in some cases, may be more likely to aggregate than larger particles. The size of the particle may affect the distribution of the particles throughout the body. For example, larger particles injected into a bloodstream may be more likely to be lodged in small vessels than smaller particles. In some instances, larger particles may be less likely to cross biological barriers (e.g., capillary walls) than smaller particles. The size of the particles used in a delivery system may be selected based on the application, and will be readily known to those of ordinary skill in the art. For example, particles of smaller size (e.g., <200 nm) may be selected if systematic delivery of the particles throughout a patient's bloodstream is desired. As another example, particles of larger size (e.g., >200 nm) may be selected if sequestering of the particles by a patient's reticuloendothelial system upon injection is desired (e.g., sequestering of the particles in the liver, spleen, etc.). The desired length of time of delivery may also be considered when selecting particle size. For example, smaller particles may circulate in the blood stream for longer periods of time than larger particles.

In some embodiments, a particle comprises a polymeric material (e.g., a polymer). A “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks. In some cases, additional moieties may also be present in the polymer, for example targeting moieties such as those described herein.

In some cases, the polymer is biologically derived, i.e., a biopolymer. Non-limiting examples include peptides or proteins (i.e., polymers of various amino acids), or nucleic acids such as DNA or RNA. In some embodiments, the polymer may be biocompatible, i.e., the polymer that does not typically induce an adverse response when inserted or injected into a living subject, for example, without significant inflammation and/or acute rejection of the polymer by the immune system, for instance, via a T-cell response. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, polycaprolactone, or copolymers or derivatives including these and/or other polymers. In some embodiments, the biocompatible polymer is biodegradable, i.e., the polymer is able to degrade, chemically and/or biologically, within a physiological environment, such as within the body. For instance, the polymer may be one that hydrolyzes spontaneously upon exposure to water (e.g., within a subject), the polymer may degrade upon exposure to heat (e.g., at temperatures of about 37° C.). Non-limiting examples of biodegradable polymers include poly(lactide) (or poly(lactic acid)), poly(glycolide) (or poly(glycolic acid)), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters) or the like, and copolymers or derivatives of these and/or other polymers, for example, poly(lactide-co-glycolide) (PLGA).

In some embodiments, the polymer may be a polymer which has been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates. In some embodiments, the polymer may be PEGylated, as described herein.

In some embodiments, method for treating a patient, such as a patient in need of therapeutic protocol (e.g., indicated for treatment for cancer) are provided. According to the first set of embodiments, a method comprises administering to a patient a complex comprising platinum compound and an inhibitor (e.g., for IDO). In some embodiments, upon uptake of the complex into a cell, the inhibitor (e.g., for IDO) dissociates from each the platinum center and a therapeutically active platinum(II) agent (or a precursor thereof) form. In some embodiments, synergistic therapeutic effect of platinum-IDO constructs exceed that of the mono-therapy agents of the corresponding platinum (II)/(IV) agent and/or the IDO inhibitor between about about 4 to about 4000 fold, or between about 10 to about 1000 fold.

In some embodiments, compositions, preparations, formulations, kits, and the like, comprising any of the complexes as described herein are provided. In some cases, treatment of a cancer may involve the use of compositions, as described herein. That is, one aspect of the invention involves a series of compositions (e.g., pharmaceutical compositions) or agents useful for treatment of cancer or tumor. These compositions may also be packaged in kits, optionally including instructions for use of the composition for the treatment of such conditions. These and other embodiments of the invention may also involve promotion of the treatment of cancer or tumor according to any of the techniques and compositions and combinations of compositions described herein.

In some embodiments, the complexes described herein may be used to prevent the growth of a tumor or cancer, and/or to prevent the metastasis of a tumor or cancer. In some embodiments, the complex may be used to shrink or destroy a cancer. It should be appreciated that complex may be used alone or in combination with one or more additional anti-cancer agents or treatments (e.g., chemotherapeutic agents, targeted therapeutic agents, pseudo-targeted therapeutic agents, hormones, radiation, surgery, etc., or any combination of two or more thereof). In some embodiments, a composition of the invention may be administered to a patient who has undergone a treatment involving surgery, radiation, and/or chemotherapy. In certain embodiments, a composition of the invention may be administered chronically to prevent, or reduce the risk of, a cancer recurrence.

In some embodiments, pharmaceutical compositions are provided comprising a complex as described herein or a pharmaceutically acceptable salt, thereof, and one or more pharmaceutically acceptable carriers, additives and/or diluents. In some embodiments, the pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, the complexes described herein useful for treatment of cancer may be packaged in kits, optionally including instructions for use of the composition for the treatment of cancer. That is, the kit can include a description of use of the complex for participation in any biological or chemical mechanism disclosed herein associated with cancer or tumor. The kits can further include a description of activity of cancer in treating the pathology, as opposed to the symptoms of the cancer. That is, the kit can include a description of use of the complex as discussed herein. The kit also can include instructions for use of a combination of two or more compositions of the invention. Instructions also may be provided for administering the drug by any suitable technique, such as orally, intravenously, or via another known route of drug delivery. In some embodiments, promotion of the treatment of cancer according to any of the techniques and compositions and composition combinations described herein is also provided.

For convenience, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

As used herein, a “subject” or a “patient” refers to any mammal (e.g., a human), such as a mammal that may be susceptible to tumorigenesis or cancer. Examples include a human, a non-human primate, a cow, a horse, a pig, a sheep, a goat, a dog, a cat, or a rodent such as a mouse, a rat, a hamster, or a guinea pig. Generally, or course, the invention is directed toward use with humans. A subject may be a subject diagnosed with cancer or otherwise known to have cancer. In certain embodiments, a subject may be selected for treatment on the basis of a known cancer in the subject. In some embodiments, a subject may be selected for treatment on the basis of a suspected cancer in the subject. In some embodiments, a cancer may be diagnosed by detecting a mutation associate in a biological sample (e.g., urine, sputum, whole blood, serum, stool, etc., or any combination thereof. Accordingly, a compound or composition of the invention may be administered to a subject based, at least in part, on the fact that a mutation is detected in at least one sample (e.g., biopsy sample or any other biological sample) obtained from the subject. In some embodiments, a cancer may not have been detected or located in the subject, but the presence of a mutation associated with a cancer in at least one biological sample may be sufficient to prescribe or administer one or more compositions of the invention to the subject. In some embodiments, the composition may be administered to prevent the development of a cancer. However, in some embodiments, the presence of an existing cancer may be suspected, but not yet identified, and a composition of the invention may be administered to prevent further growth or development of the cancer.

It should be appreciated that any suitable technique may be used to identify or detect mutation and/or over-expression associated with a cancer. For example, nucleic acid detection techniques (e.g., sequencing, hybridization, etc.) or peptide detection techniques (e.g., sequencing, antibody-based detection, etc.) may be used. In some embodiments, other techniques may be used to detect or infer the presence of a cancer (e.g., histology, etc.).

The presence of a cancer can be detected or inferred by detecting a mutation, over-expression, amplification, or any combination thereof at one or more other loci associated with a signaling pathway of a cancer.

As used herein, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “alkenyl” and “alkynyl” refer to unsaturated aliphatic groups analogous in length and possible substitution to the alkyls described above, but that contain at least one double or triple bond respectively.

The terms “heteroalkenyl” and “heteroalkynyl” refer to unsaturated aliphatic groups analogous in length and possible substitution to the heteroalkyls described above, but that contain at least one double or triple bond respectively.

As used herein, the term “halogen” or “halide” designates —F, —Cl, —Br, or —I.

The terms “carboxyl group,” “carbonyl group,” and “acyl group” are recognized in the art and can include such moieties as can be represented by the general formula:

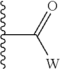

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. In some cases, the

The term “alkoxy” refers to the group, —O-alkyl.

The term “aryloxy” refers to the group, —O-aryl.

The term “acyloxy” refers to the group, —O-acyl.

The term “aralkyl” or “arylalkyl,” as used herein, refers to an alkyl group substituted with an aryl group.

The terms “heteroaryl” refers to aryl groups comprising at least one heteroatom as a ring atom.

The term “heterocycle” refers to refer to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. The heterocycle may also be fused to a spirocyclic group. In some cases, the heterocycle may be attached to a compound via a nitrogen or a carbon atom in the ring.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamnethyleneimine), piperazine (e.g., N-methyl piperazine), morpholine, lactones, lactams such as azetidinones and pyrrolidinones, sultams, sultones, other saturated and/or unsaturated derivatives thereof, and the like. The heterocyclic ring can be optionally substituted at one or more positions with such substituents as described herein. In some cases, the heterocycle may be bonded to a compound via a heteroatom ring atom (e.g., nitrogen). In some cases, the heterocycle may be bonded to a compound via a carbon ring atom. In some cases, the heterocycle is pyridine, imidazole, pyrazine, pyrimidine, pyridazine, acridine, acridin-9-amine, bipyridine, naphthyridine, quinoline, benzoquinoline, benzoisoquinoline, phenanthridine-1,9-diamine, or the like.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence. An example of a substituted amine is benzylamine. Another non-limiting example of an amine is cyclohexylamine.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and can not be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like.

The following examples are intended to illustrate certain embodiments of the present invention, but do not exemplify the full scope of the invention.

### Example 1

### Summary

Expression of indoleamine-2,3-dioxygenase (IDO), an immunosuppressive enzyme in human tumors, leads to immune evasion and tumor tolerance. IDO is therefore a tumor immunotherapeutic target, and several IDO inhibitors are currently undergoing clinical trials. IDO inhibitors can enhance the efficacy of common cancer chemotherapeutics. Pt(IV)-(D)-1-methyltryptophan conjugates 1 and 2 were investigated for combined immunomodulation and DNA cross-link-triggered apoptosis for cancer ‘immuno-chemotherapy’. Compound 2 effectively killed hormone-dependent, cisplatin-resistant human ovarian cancer cells, inhibiting IDO by transcriptional deregulation of the autocrine-signaling loop IDO-AHR-IL6, which blocks kynurenine production and promotes T-cell proliferation. Additionally, 1 and 2 displayed low toxicity in mice and are stable in blood.

### Discussion

Attractive immunotherapy approaches have included chimeric antigen receptor (CAR) T-cell therapies, cancer vaccines, dendritic cell therapies, and immune checkpoint inhibitors. Immune checkpoint therapy has become a clinically viable treatment alternative to conventional chemotherapy for cancer following the FDA approval of ipilumumab, pembrolizumab, and nivolumab. Several immune checkpoints are involved in tumor immune escape with varied biological functions, signaling pathways, and expression levels in tumors. The programmed death (PD-1), cytotoxic T-lymphocyte antigen CTLA, T-cell immunoglobulin and mucin 3 domain (TIM3), and IDO are common inhibitory immune checkpoint targets under investigation. Immune checkpoint therapy targets regulatory pathways that affect T-cells to enhance antitumor immune responses. Combining this therapy, by using small molecule immune checkpoint inhibitors, with standard chemotherapy may provide survival benefit to patients.

IDO is a heme-containing oxidoreductase encoded by the INDO gene. IDO catalyzes the degradation of the essential amino acid tryptophan to kynurenine with the exception of dietary tryptophan, which is catabolized by the liver enzyme tryptophan dioxygenase (TDO). The depletion of tryptophan mediates immune tolerance by suppressing effector T-cell function through G1 arrest and subsequent inactivation. In a variety human tumors and host antigen-presenting cells, elevated levels of IDO are characteristic of poor prognosis. Small molecule inhibitors of IDO that stimulate antitumor immunity have emerged with (D)-1 methyltryptophan ((D)-1-MT) and INCB-24367 in Phase I/II clinical trials for the treatment of breast, brain, melanoma, and pancreatic cancers. Promising IDO inhibitors with unique chemical scaffolds continue to attract attention, among which include brassinins, quinones, phenylimidazoles, and hydroxy-amidines. These small molecules have the advantage of being easy to produce and deliver, low cost, and compatible with conventional cancer therapies.

IDO inhibitors enhance the efficacy of common chemotherapeutics and are synergistic with radiation therapy. The IDO inhibitor methylthiohydantoin-tryptophan (MTH-Trp) in combination with cisplatin regresses autochthonous murine breast tumors. Induction of IDO-blockade using (D)-1-MT and NLG919 works synergistically with temozolomide (TMZ), cyclophosphamide, and radiotherapy to treat GL261 tumors (glioblastoma) in a syngeneic mouse model. Combination chemotherapy incorporating IDO inhibitors holds promise for cancer therapy. A dual—threat construct having a potent chemotherapeutic and immune checkpoint inhibitor has thus far not been reported.

Platinum-based chemotherapy is first line treatment for many cancers in the clinic. The FDA-approved Pt agents include cisplatin, carboplatin, and oxaliplatin. They induce apoptosis in cancer cells, primarily through DNA damage. Despite the efficacy of Pt drugs, toxicity, tumor recurrence, acquired and inherent resistance, and deactivation are associated drawbacks that remain problematic. To overcome these problems, one chemical strategy that may be employed has been to design an inert Pt(IV) prodrug that can be activated by intracellular reduction following cellular uptake. Given the aforementioned limitations of conventional chemotherapy and immunotherapy, and taking advantage of the potential synergy between platinum drugs and immune checkpoint inhibitors, this example employs the Pt(IV) prodrug strategy to combine immunomodulation with Pt-DNA cross-linking-induced apoptosis, affording an effective chemo-immunotherapeutic.

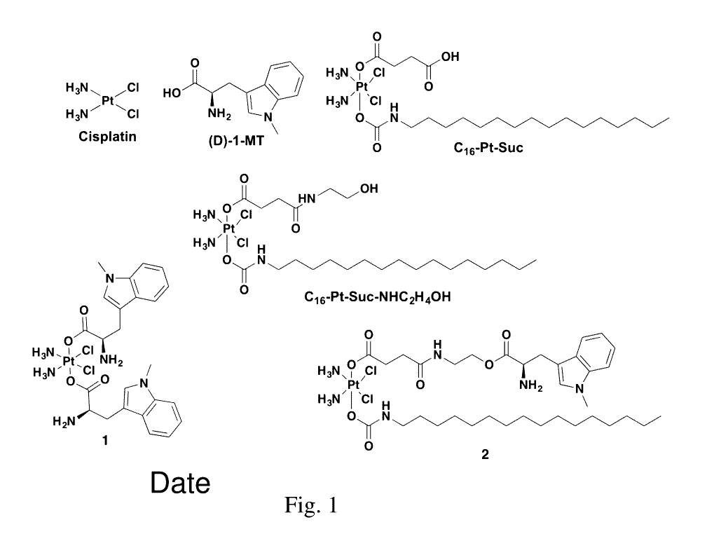

A symmetric manifestation of the design attached two (D)-1-MT units at the axial positions of a cisplatin pro-drug (1, FIG. 1). An asymmetric construct having a hexadecyl hydrophobic chain at one axial position and (D)-1-MT at the other was also prepared (2, FIG. 1). The latter synthetic strategy provides a unique double prodrug, activated both by intracellular reduction and by esterase activity. Detailed chemical studies revealed that 2 binds HSA, as evidenced by FPLC, graphite furnace atomic absorption spectrophotometry (GFAAS), and ESI mass-spectrometric analysis. Furthermore, the degree of lipophilicity of (D)-1-MT, 1 and 2 was determined by measuring the extent of compound partition between octanol and water, P_{o/w}. Experimentally determined Log P values increased from (D)-1-MT (Log P=−2.98±0.15), to 1 (Log P=−0.21±0.08) to 2 (Log P=1.35±0.26), which may affect their cellular differential uptake. Without wishing to be bound by theory, the release of (D)-1-MT from 1 or 2 inside cancer cells may block IDO to prevent T-cell degradation, while generation of cisplatin could concomitantly induce DNA damage-induced cell death. A thorough investigation of this novel, dual-threat prodrug approach provided the results presented here, including the synthesis and characterization, cellular mechanism, functional IDO inhibition, and cellular immunomodulation of 1 and 2. The potent anti-proliferative ability of the constructs, nanoparticle formulation, and stability in mice were also investigated.

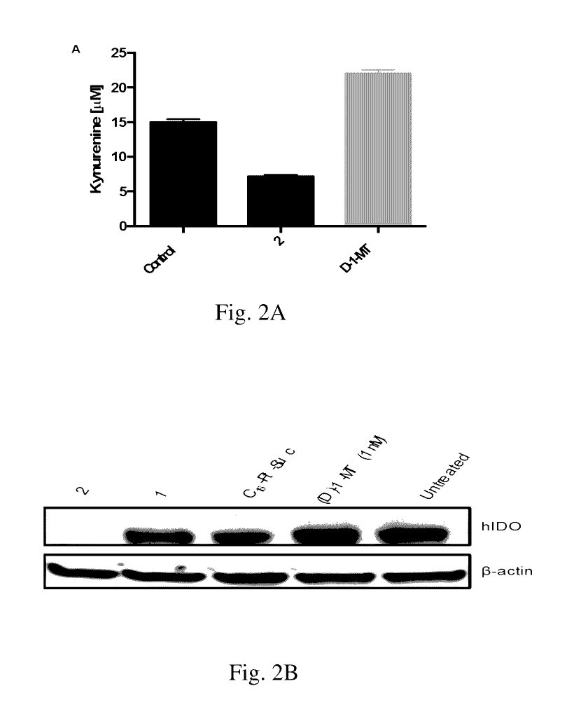

Compounds 1 and 2 were prepared and characterized as outlined in Example 2 and tested against a panel of human ovarian cancer cells with varying sensitivities to cisplatin and constitutive expression of IDO. It was found that SKOV3 cells constitutively express robust levels of IDO, A2780 cells express the protein minimally, and NIH:OVCAR3 not at all. The conversion of tryptophan to kynurenine by IDO suppresses the anti-tumor immune response. Therefore, the ability of 1 and 2 to block kynurenine production in cells was investigated. With the use of HPLC, 2 blocks kynurenine release in the medium of SKOV3 cultured cells by a factor of three in comparison to treatment with (D)-1-MT (FIG. 2A). Using immunoblotting and qRT-PCR, 2 was found to selectively target and effectively block IDO protein expression (FIG. 2B) while 1 does so at a 10-fold higher concentration (100 μM). Previously it was demonstracted that cisplatin-DNA adducts block transcription, ultimately triggering apoptosis. However, investigation of the control platinum construct C₁₆—Pt-Suc did not show IDO inhibition relative to 2 (FIG. 2B). This result indicates that Pt-DNA adducts may not block IDO transcription or induce IDO mRNA instability, but (D)-1-MT released in the cells may. Collectively, these findings demonstrate the ability of 2 to interrupt the kynurenine pathway by depleting IDO.

The ability of 1, 2, and cisplatin as a control to promote cell death was evaluated by the MTT [3-(4,5-dimethylthiazol-2-yl)-2,5-diphenyl-tetrazolium bromide] assay across a panel of human ovarian cancer cells. The concentration required for inducing 50% cell kill (IC₅₀) ranged from high nanomolar to micromolar, as extrapolated from dose-response curves; the results are summarized in Table 1. Significantly, complex 2 was the most potent with no cross-resistance with cisplatin as typified by its ability to induce cell death in both cisplatin-sensitive and resistant ovarian cancer cell lines A2780 and A2780/CP70, SKOV3 and NIH:OVCAR3. 2 displayed approximately 2-4000-fold potency in IDO expressing cells, A2780 and SKOV3 over cisplatin, co-incubated [cisplatin+(D)-1MT] and C₁₆—Pt-Suc-NHC₂H₄OH treated cells (Table 2).

TABLE 1
Experimental Log P of 1, 2 and (D)-1-MT. The Log P was determined
by measuring the extent of partition between octanol and water

| | Compound | | |
|---|---|---|---|
| | 1 | 2 | (D)-1-MT |
| Log P | −0.21 ± 0.08 | 1.35 ± 0.26 | −2.98 ± 0.15 |

TABLE 2
IC₅₀ values (in (μM) of conjugates 1, 2, cisplatin, and relevant control compounds against a panel of human
ovarian cancer cell lines. For co-incubation of cisplatin and (D)-1-MT, cells were pretreated with 1 mM (D)-1-MT

| | Cancer | 1 | 2 | Cisplatin | (D)-1-MT | Cisplatin + (D)-1-MT | C₁₆-Pt-Suc-NHC₂H₄OH |
|---|---|---|---|---|---|---|---|
| Cell line | type | (μM) | (μM) | (μM) | (μM) | (μM) | (μM) |
| A2780 | Ovarian | 0.98 ± 0.01 | 0.20 ± 0.08 | 0.70 ± 0.20 | 833 ± 7.53 | 0.89 ± 0.20 | 1.34 ± 0.13 |
| A2780/CP70 | Ovarian | 8.50 ± 0.02 | 0.28 ± 0.04 | 10.4 ± 0.60 | 1045 ± 63.85 | 6.20 ± 1.28 | 2.30 ± 0.35 |
| NIH:OVCAR3 | Ovarian | 2.39 ± 0.04 | 1.37 ± 0.03 | 4.10 ± 0.20 | 1198 ± 80.01 | 2.52 ± 0.90 | 1.33 ± 0.12 |
| SKOV3 | Ovarian | 3.76 ± 0.08 | 1.02 ± 0.11 | 3.70 ± 0.28 | 1918 ± 8.40 | 3.8 ± 0.54 | 1.32 ± 0.12 |

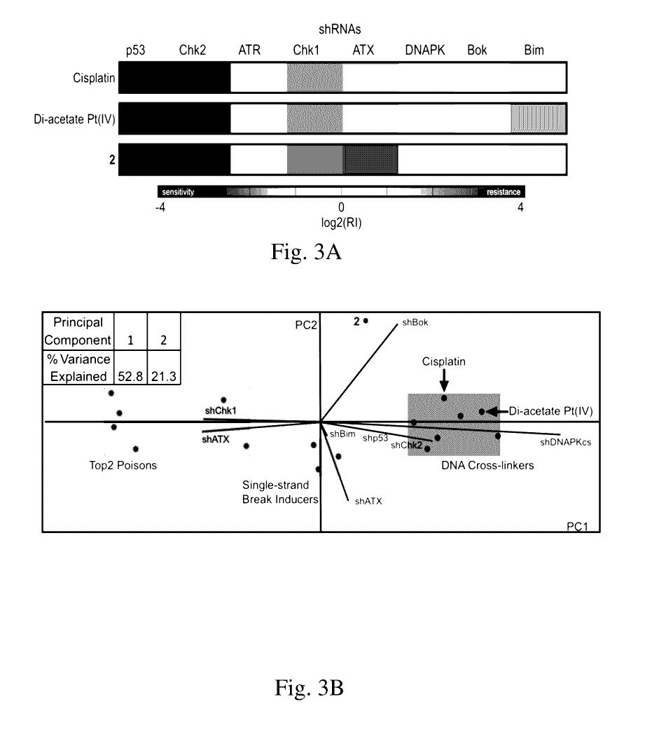

The intracellular behavior of the Pt(IV)-(D)-1-MT construct (2) was analyzed by using an RNA interference (RNAi) methodology and cell cycle analysis. This mechanism of action predictive RNAi methodology uses lymphoma cells that are partially infected with one of eight GFP-labelled short hairpin RNAs (shRNAs). Each shRNA has the potential ability to confer resistance or sensitivity to a particular drug depending on its mechanism of action. Thus, after treatment with the drugs of interest, the cells are subjected to flow cytometry to assess GFP percentage. The resulting pattern of resistance and sensitivity to a given drug is then processed by a probabilistic K-nearest neighbors algorithm that assigns novel compounds to a category by comparing their signatures to that of reference set of drugs. The RNAi signatures of cisplatin, cis,cis,trans-[Pt(NH₃)₂Cl₂(OCOCH₃)₂] (Di-acetate Pt(IV)), and 2 were obtained. Interestingly, although 2 displays features of a DNA damaging agent, such as strong p53 and Chk2 shRNA enrichment, it differs significantly in other shRNAs, as shown in FIG. 3A. In addition, DNA content assessment by flow cytometry revealed that 2 induces a cell cycle block at G1 phase in a time dependent fashion, which deviates from the behavior of the classical G2/M arrest phase imposed by cisplatin. The collective evidence from both RNAi signature assays and cell cycle analysis indicates a unique mechanism of action triggered by the activity of (D)-1-MT provided by the construct (FIG. 3B).

The ability of 2 to induce DNA damage was investigated by monitoring changes in expression of markers of the damage response pathway by immunoblotting analyses. SKOV3 cells incubated with 2 for up to 48 h and 72 h showed a marked time-dependent increase in expression of phosphorylated ATR, ATM, BRCA1, Chk1, Chk2, H2AX, and p53, indicative of DNA damage. Notably, the high phosphoBRCA1 response confirms the DNA damage role of BRCA1 specific to ovarian and breast cancer.

To gain further insight into the mode of action was studied of 1 and 2, the subcellular distribution of platinum. SKOV3 cells were incubated with the drug candidates (5 μM) for 17 h, using cisplatin as a control. The platinum content was measured by using GFAAS. Whereas the platinum content within the nucleus following 2 treatment was comparable to that of cisplatin, it was 7-fold higher in the cytoplasm. This result indicates that, over the duration of the study, 2 is preferentially taken up over both 1 and cisplatin, possibly due to its lipophilic axial chain. In addition, DNA platination studies revealed the occurrence of 310±75 Pt adducts/10⁴ nucleotides for 1, 409±54 Pt adducts/10⁴ nucleotides for cisplatin and 970±110 adducts/10⁴ nucleotides for 2. These results demonstrate that, like cisplatin, the new platinum constructs target genomic DNA.

Mechanistic studies of the effect of 2 on the IDO pathway were performed by examining the autocrine-signaling loop of IDO-AHR-IL6 (Figure S20). The activation of AHR, a cytosolic transcription factor that translocates to the nucleus upon binding xenobiotic ligands such as kynurenine, is involved in tumor formation. The signal transducer and activator of transcription STAT3 mediates the process of carcinogenesis. Without wishing to be bound by theory, 2, after spontaneous intracellular reduction and esterase activation may release (D)-1MT into the cytosol to block IDO, leading to inactivation of AHR owing to kynurenine inhibition. Recent findings show that interleukin 6 (IL-6) modulates IDO expression via STAT3. To support this hypothesis, SKOV3 cells were treated with 2 and the RNA was harvested after 24 h for qPCR. The results show that mRNA expression levels of AHR decreased by 5-fold and those of IL-6 were reduced by 10-fold relative to untreated controls. These results support the role of 2 to interrupt the IDO pathway by deactivating the downstream AHR and IL-6, thereby destabilizing the autocrine loop implicated in constitutive IDO expression.

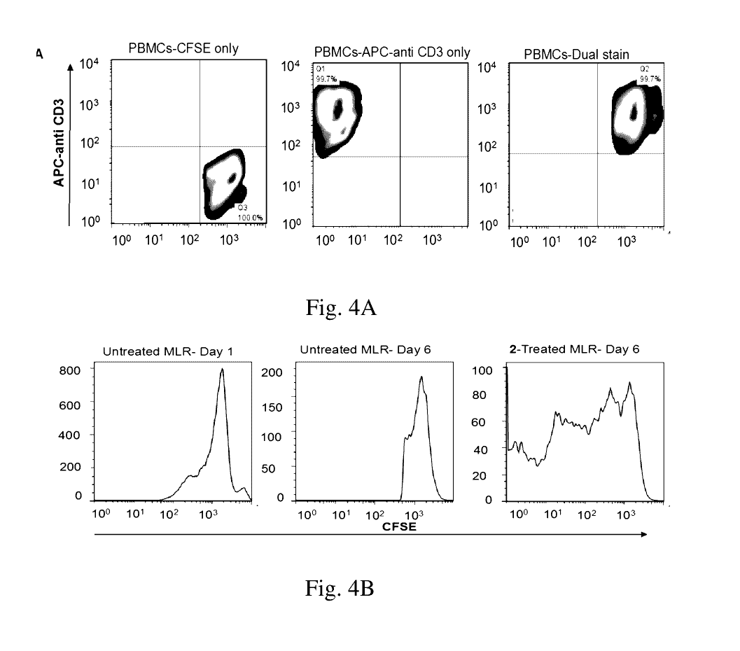

To target a key component of the immunosuppression promoting IDO-AHR-IL6 loop, the immunomodulatory phenotype of 2 was investigated. A mixed leukocyte reaction (MLR) was carried out. In this experiment, suspended 2×10⁵ peripheral blood mononuclear cells (PBMCs) stimulated with phytohemagglutinin (PHA-P), and 2×10⁵ cells of PBMCs from unrelated healthy donors as responders stained with carboxyfluorescein succinimidyl ester (CFSE), a cell permeable fluorescent dye that binds amine residues (mostly lysines) covalently, through its succinimidyl ester group were cocultured with adherent SKOV-3 (2000 cells). After 6 days of culture, PBMCs were harvested by centrifugation of medium, stained with APC-anti CD3 and T-cell proliferation was assessed by flow cytometry. T-cells express CD3, a cell surface multimeric protein complex involved in T-cell development and activation. PBMCs used were CD3^{high} as assessed by flow cytometry using APC labeled anti-CD3, as shown in FIG. 4A. Consistent with the results of a decrease in kynurenine production by 2 due to downregulation of IDO and its downstream targets, the presence of 2 in the SKOV3/MLR coculture experiment resulted in the enhancement of alloreactive T-cell proliferation assessed by live-cell tracing for multiple generations using flow cytometry (FIG. 4B). Collectively, the cellular data show that 2 plays a role in immunomodulation, which results in T-cell proliferation.

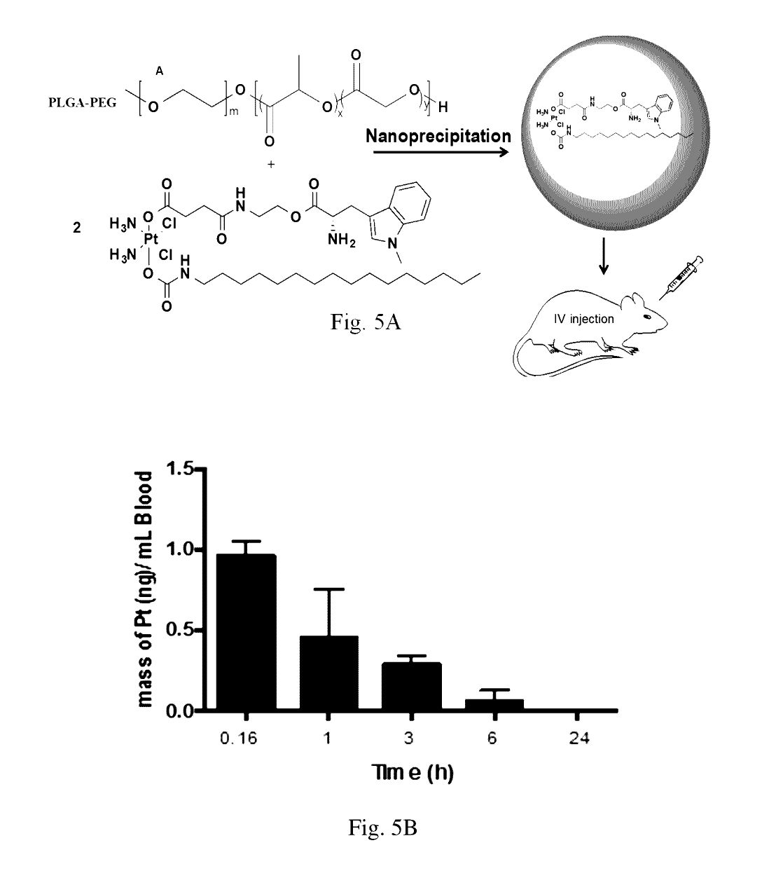

Therapeutic nanoparticles (NPs) have been widely investigated for their potential to enhance cancer treatment. The chemical structure of 2 bears both hydrophilic (1-D-MT) and hydrophobic (hexadecyl isocyanate) axial ligands, conferring amphiphilic character to the molecule. In some cases, the hexadecyl isocyanate axial ligand facilitate non-covalent binding to human serum albumin, protecting the Pt(IV) center from premature reduction in blood. In the presence of the biodegradable block copolymer, poly(lactic-co-glycolic acid)-polyethylene glycol (PLGA-PEG), 2 readily self-assembles into NPs suitable for preclinical studies (FIG. 5A). Characterization of the NPs using DLS showed sizes of 120±3.50 nm, applicable for preclinical studies. Having obtained the nanoparticles, the ability of 2 and its NP to induce apoptosis was investigated. Translocation of phosphatidylserine residues to the exterior is a characteristic of apoptotic cells, which have a compromised cell membrane, and can be detected by annexin V. Using a dual staining annexin V/sytox green apoptosis dead cell assay, we examined the apoptotic behavior of SKOV3 cells after 72 h of treatment with 2 and its NP by flow cytometry. Both 2 and its NP induced large populations of SKOV3 cells to undergo apoptosis.

In vivo evaluation of 2 revealed that mice are tolerant of the platinum-immune checkpoint inhibitor conjugates. Dynamic blood stability experiments of 2 in six-week old female Balb/c mice revealed a t_{1/2} of approximately 1 h, as shown in FIG. 5B. Mice were intravenously injected with a sterile NP formulation of 2 at a relatively high dose of 8 mg/kg and blood was drawn at different time intervals (10 min, 1 h, 3 h, 6 h and 24 h). The hydrophobic complex 2 was extracted with octanol, diluted, and the Pt concentration was determined by GFAAS. By 24 h the Pt concentration reached non-detectable levels suggesting complete reduction of hydrophobic Pt(IV) to hydrophilic Pt(II) species. In addition, blood stability in human blood showed that the t₁₁₂ of 2 is 2.2 h in comparison to that reported for cisplatin (t_{1/2}=21.6 min) and satraplatin (t_{1/2}=6 min).

In conclusion, a Pt small molecule immune checkpoint inhibitor platform that targets the immunosuppressive enzyme IDO to enhance T-cell proliferation was exemplified. The synergy of Pt and checkpoint inhibitor biology as well as the molecular mechanism of (D)-1-MT was investigated. The cellular data provide evidence for DNA damage induced by 2, which can lead to G1 arrest and cell death. Furthermore, IDO is inhibited to evoke downregulation of AHR and IL-6, which are key genes involved in the auto-regulation of constitutive IDO expression. This action leads to immunomodulation and enhanced T-cell proliferation.

### Example 2

The following example provide additional details regarding the material and methods in connection with Example 1.

### Experimental Details

### General Information

Compounds 1 and 2 were prepared as shown in Scheme 1. Known intermediates were prepared following reported protocols (e.g., see Zheng, Y. R.; Suntharalingam, K.; Johnstone, T. C.; Yoo, H.; Lin, W.; Brooks, J. G.; Lippard, S. J. J. Am. Chem. Soc. 2014, 136, 8790. Dhar, S.; Daniel, W. L.; Giljohann, D. A.; Mirkin, C. A.; Lippard, S. J. J. Am. Chem Soc. 2009, 131, 14652). All reagents were purchased from Strem, Sigma Aldrich, or Alfa Aesar and used without further purification, including anhydrous DMF and DMSO. All reactions were carried out under normal atmospheric conditions. Deuterated solvents were purchased from Cambridge Isotope Laboratories (Andover, Mass.). ¹H, ¹³C and ¹⁹⁵Pt NMR spectra were recorded on a Varian Unity 300/500 NMR spectrometer with a Spectro Spin superconducting magnet in the Massachusetts Institute of Technology Department of Chemistry Instrumentation Facility (MIT DCIF). Chemical shifts in ¹H and ¹³C NMR spectra were internally referenced to solvent signals (¹H NMR: DMSO at 6=2.50 ppm; ¹³C NMR: DMSO at 6=40.45 ppm), and those in ¹⁹⁵Pt NMR spectra were externally referenced to K₂PtCl₄ in D₂O (δ=−1628 ppm). Electrospray ionization mass spectrometry (ESI-MS) was performed on an Agilent Technologies 1100 series liquid chromatography/MS instrument. High-resolution mass spectra (HRMS) were obtained by direct flow injection (injection volume=5 or 2 μL) ElectroSpray Ionization (ESI) on a Waters Qtof API US instrument in the positive mode (CIC, Boston University). Typical conditions are as follows: capillary=3000 kV, cone=35 or 15, source temperature=120° C., and desolvation temperature=350° C. Analytical HPLC was conducted on an Agilent 1200 system using C18 reverse phase columns (Agilent Eclipse XDBC18, 4.6 mm×250 mm, 5 μm; Vydac® 218TP5415, 4.6 mm×250 mm, 5 μm). Graphite furnace atomic absorption spectroscopic (GFAAS) measurements were taken on a Perkin Elmer AAnalyst 600 spectrometer. Distilled water was purified by passage through a Millipore MilliQ Biocel water purification system (18.2 mV) equipped with a 0.22 μm filter. Flow cytometry experiments were performed at the MIT flow cytometry core facility. PBMCs from two different donors were purchased from Zen-Bio, Inc. (Research Triangle, NC). Single donor human whole blood with Li-heparin was purchased from Innovative Research (Novi, Mich.). The blood sample was kept at 4° C. and used within three days of receipt. Female Balb/c mice were purchased from Charles River laboratory and the animal experiments were performed at the Koch Institute animal facility with approval from the MIT Committee for Animal Care (CAC).

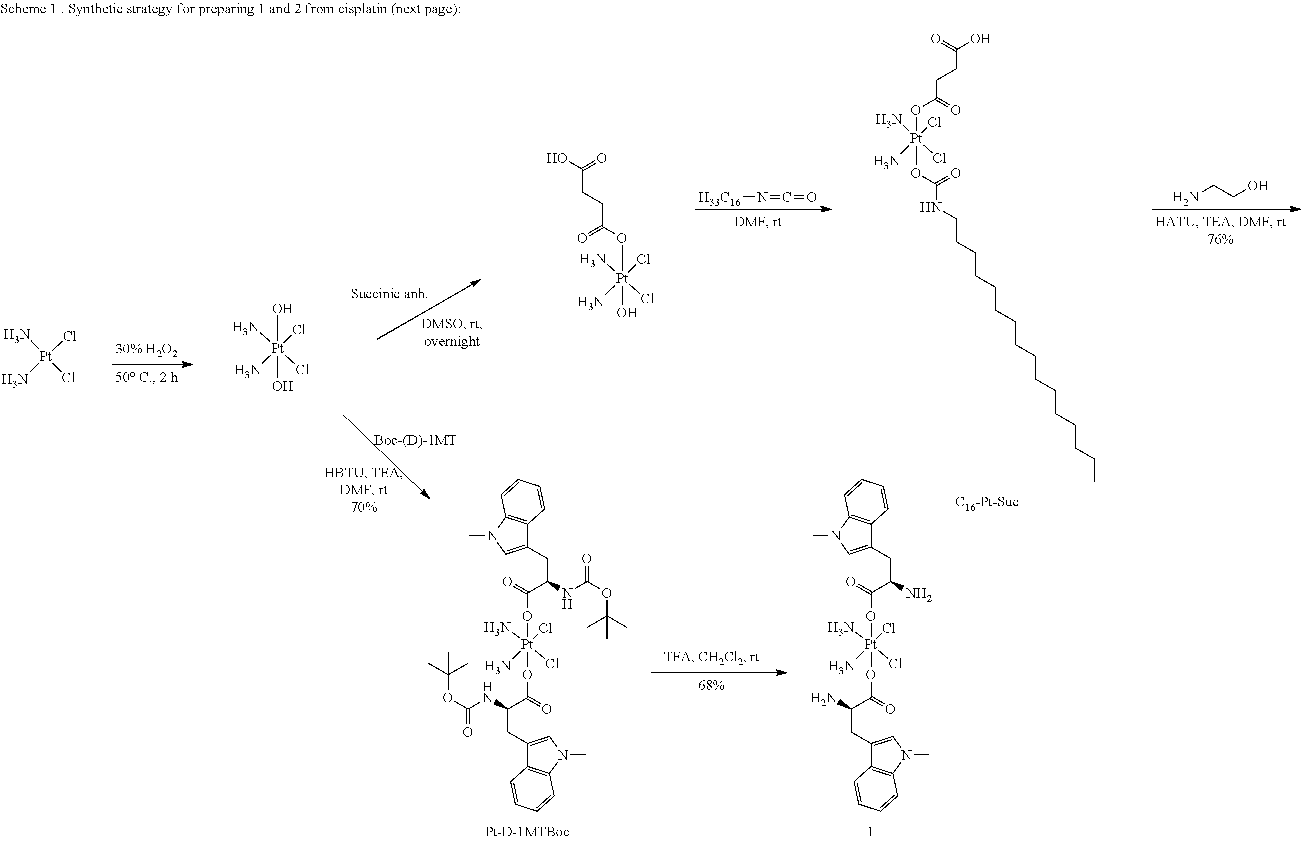

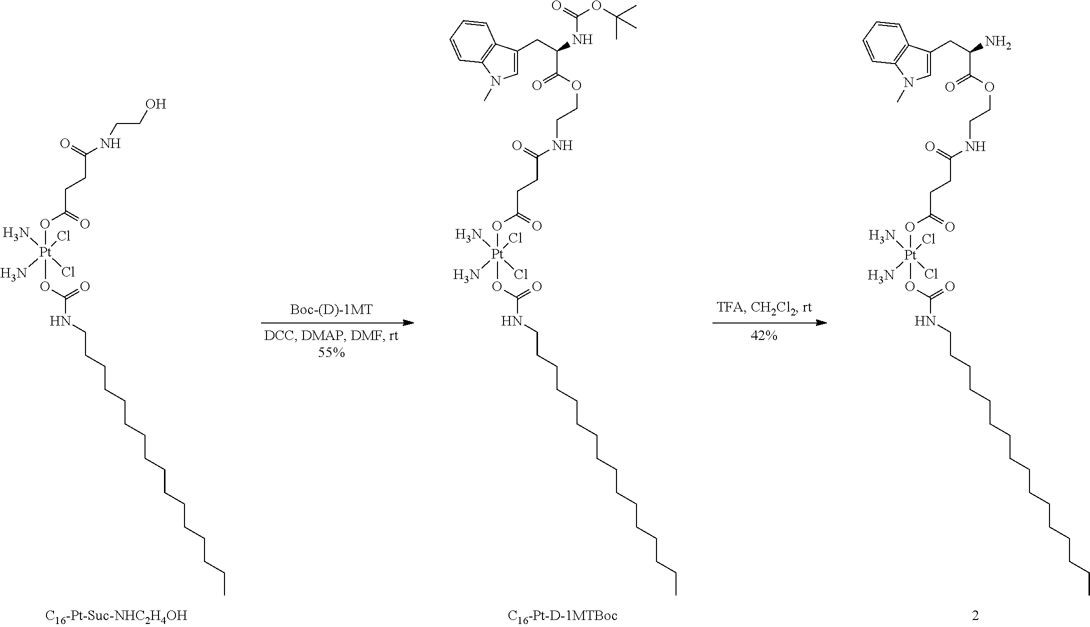

### N-Boc-1-methyl-D-tryptophan (Boc-D-1MT)

D-1-methyltryptophan (250 mg, 1.14 mmol), NaHCO₃ (288.70 mg, 3.44 mmol), and di(tert-butyl) dicarbonate (300 mg, 1.37 mmol) were dissolved in an equal volume of THF:H₂O (20 mL) and the mixture was stirred at 0° C. for 10 min and then at room temperature for 24 h. THF was evaporated and the aqueous layer was acidified with 1 M HCl (10 mL) to pH 1 and then extracted with ethyl acetate (15 mL, 3×). The ethyl acetate was evaporated to leave a pure pale white solid. Yield: 270 mg, 74%. ¹H NMR (300 MHz, CD₂Cl₂, ppm): δ 7.58 (d, J=9.0 Hz, 1H, Ar—H), 7.32 (d, J=9.0 Hz, 1H, Ar—H), 7.25-7.19 (m, 1H, Ar—H), 7.12-7.07 (m, 1H, Ar—H), 6.94 (s, 1H, C—H_{N-methylindole}), 5.09 (s, 1H, NH), 4.60 (s, 1H, CH), 3.70 (s, 3H, N—CH₃), 3.31 (s, 2H, CH₂), 1.41 (s, 9H, C(CH₃)₃)_{.} ¹³C NMR (75 MHz, CD₂Cl₂, ppm): δ 174.6, 156.1, 137.2, 121.7, 119.1, 110.2, 78.7, 55.3, 32.9, 28.8. ESI-MS (positive mode) C₁₇H₂₂N₂O₄: [2M+Na]⁺, cald, m/z 659.2; found, 659.2.

### cis,cis,trans-[Pt(NH₃)₂Cl₂(O₂C(Boc)-D-1-MT)₂] (Pt-D-1MTBoc)

To a suspension of cis,cis,trans-[Pt(NH₃)₂(Cl₂)(OH)₂] (35 mg, 0.10 mmol) in DMF was added Boc-D-1MT (100 mg, 0.31 mmol), HBTU (119 mg, 0.31 mmol), and triethylamine (1 equiv). The mixture was stirred for 24 h. The resulting solution was filtered, and the filtrate was reduced to slurry under high vacuum (temperature=65° C.). The residue was dissolved in ethanol and the compound was precipitated with water. The yellow residue was purified by silica-gel column chromatography (acetone/hexane=30:70 v/v) to collect a yellow solution, which was dried to afford a yellow solid. Yield: 57 mg, 70%. ¹H NMR (300 MHz, CD₂Cl₂, ppm): δ 7.59 (d, J=9.0 Hz, 2H, ArH), 7.33 (d, J=9.0 Hz, 2H, ArH), 7.25-7.19 (m, 2H, ArH), 7.12-7.07 (m, 2H, ArH), 6.94 (s, 2H, C—H_{N-methylindole}), 5.04 (s, 1H, NH), 4.60 (s, 2H, CH), 3.75 (s, 6H, N—CH₃), 3.31 (s, 4H, CH₂), 1.41 (s, 18H, C(CH₃)₃). ¹³C NMR (125 MHz, DMSO-d₆, ppm): δ 180.7, 175.0, 156.2, 137.2, 137.6, 128.9, 121.9, 120.1, 119.5, 110.1, 78.7, 46.3, 29.0, 9.1 ESI-MS (positive mode)C₃₄H₄₈Cl₂N₆O₈Pt: [M+Na]⁺, cald, (m/z) 957.3; found, 957.3.

### cis,cis,trans-[Pt(NH₃)₂Cl₂(O₂C-D-1-MT)₂]. (1). Pt-D-1MTBoc

(50.0 mg, 0.05 mmol) was suspended in 10% TFA/CH₂Cl₂ v/v (1 mL) at room temperature for 1 h, after which the solution was air-dried and dissolved in a minimum amount of CH₂Cl₂ (0.5 mL) and added dropwise to a large volume (10 mL) of diethyl ether. The pale yellow solid was collected by centrifugation and washed with diethyl ether, three times. Yield: 25 mg, 68%. ¹H NMR (300 MHz, DMF-d₇, ppm): δ 8.78 (br. s, 4H, NH₂), 7.68 (d, J=9.0 Hz, 2H, ArH), 7.45 (d, J=9.0 Hz, 2H, ArH), 7.32 (s, 2H, C—H_{N-methylindole}), 7.25-7.20 (m, 2H, ArH), 7.18-7.08 (m, 2H, ArH), 4.49-4.44 (m, 1H, CH), 4.30-4.25 (m, 1H, CH), 3.55 (s, 6H, N—CH₃), 3.51-3.43 (m, 2H, CH₂), 3.27-3.22 (m, 2H, CH₂). ¹³C NMR (125 MHz, DMSO-d₆, ppm): δ 171.9, 137.7, 130.2, 128.5, 122.4, 119.8, 110.8, 107.6, 54.1, 33.5, 27.2. ¹⁹⁵Pt NMR (108 MHz, DMSO-d₆, ppm) δ 1244.8. HRMS (positive mode) C₂₄H₃₂Cl₂N₆O₄Pt: [M+H], cald, (m/z) 735.1577; found, 735.1573. Anal. calcd for C₂₄H₃₂Cl₂N₆O₄Pt.0.3H₂O: C, 39.24; H, 4.39; N, 11.44, found: C, 38.96; H, 4.44; N, 11.36.

### cis,cis,trans-[Pt(NH₃)₂Cl₂(O₂CC₂H₄OCNHC₂H₄OH)(O₂CNHC₁₆H₃₃)](C₁₆—Pt-Suc-NHC₂H₄OH)

To a suspension of C₁₆—Pt-Suc (100 mg, 0.14 mmol) in DMF (4 mL) was added HATU (108 mg, 0.28 mmol), ethanolamine (17 μL), and triethylamine (39 μL). The reaction turned bright yellow and was allowed to stir at room temperature overnight. The mixture was subsequently filtered and DMF was removed in vacuo. The residue was washed with diethyl ether to obtain a pale yellow solid. Yield: 136.8 mg, 76%. ¹H NMR (500 MHz, DMSO-d₆, ppm): δ 7.80 (s, 1H, NH), 6.56 (s, 6H, NH₃), 4.62 (s, 1H, OH), 3.07 (s, 2H, CH₂), 2.85 (s, 2H, CH₂), 2.40 (s, 2H, CH₂), 2.23 (s, 2H, CH₂), 1.22 (s, 28H, CH₂), 0.82 (t, 3H, CH₃). ¹³C NMR (125 MHz, DMSO-d₆, ppm): δ 181.0, 172.7, 162.7, 61.0, 47.9, 45.7, 42.2, 32.9, 30.1, 27.2, 23.2, 9.5. ESI-MS (positive mode) C₂₃H₅₀Cl₂N₄O₆Pt: [M+H]⁺, cald, (m/z) 744.2; found, 744.3.

### cis,cis,trans-[Pt(NH₃)₂Cl₂(O₂CC₂H₄OCNHC₂H₄O₂C(Boc)-D-1MT)(O₂CNHC₁₆H₃₃)] (C₁₆—Pt-D-1MTBoc)

To a stirring solution of Boc-D-1MT (22 mg, 0.07 mmol) in anhydrous DMF (3 mL) was added DMAP (0.5 mg) and C₁₆—Pt-Suc-NHC₂H₄OH (50 mg, 0.07 mmol). DCC (16 mg, 0.08 mmol) was added to the reaction mixture at 0° C. and stirring continued for an additional 5 min. The reaction was warmed to room temperature and then stirred overnight. Excess urea was precipitated by the addition of dichloromethane and filtered off to leave a clear filtrate, to which was added pentane to precipitate a yellow solid. Yield: 41 mg, 55%. ¹H NMR (500 MHz, DMSO-d₆, ppm): δ 7.98 (s, 1H, NH), 7.53-7.50 (m, 1H, ArH), 7.35-7.30 (m, 2H, ArH), 7.18-7.10 (m, 2H, NH, CH_{N-methylindole}), 6.98-6.97 (m, 1H, ArH), 6.61 (s, 6H, NH₃), 4.16 (s, 1H, CH), 4.10-4.00 (m, 2H, CH₂), 3.72 (s, 3H, N—CH₃), 3.22 (s, 2H, CH₂), 2.89 (s, 2H, CH₂), 2.44 (s, 2H, CH₂) 2.27 (s, 2H, CH₂), 1.69 (s, 2H, CH₂CH₂), 1.55 (s, 2H, CH₂CH₂), 1.44 (s, 2H, CH₂CH₂), 1.30 (s, 9H, C(CH₃)₃), 1.22 (s, 22H, CH₂(CH₂)₁₁CH₃), 0.83 (t, 3H, CH₂(CH₂)₁₃CH₃). ¹³C NMR (125 MHz, DMSO-d₆, ppm): δ 174.95, 173.4, 162.3, 156.6, 140.3, 138.0, 128.8, 122.5, 119.68, 110.7, 110.1, 108.0, 79.5, 63.7, 55.3, 46.2, 32.4, 30.7, 29.0, 23.2, 9.5. ESI-MS (negative mode) C₄₀H₇₀Cl₂N₆O₉Pt: [M−H]⁻, cald, (m/z) 1044.4; found, 1044.7.

### cis,cis,trans-[Pt(NH₃)₂Cl₂(O₂CC₂H₄OCNHC₂H₄O₂C-D-1MT)(O₂CNHC₁₆H₃₃)] (2)

To a solution of C₁₆—Pt-D-1MTBoc (35 mg, 0.03 mmol) in dichloromethane (0.9 mL) was added TFA to a final concentration of 10% v/v. The mixture was stirred at room temperature for 4 h and then filtered. The filtrate was air dried, re-dissolved in a minimum amount of dichloromethane (0.5 mL), and added dropwise to a large volume of diethyl ether (10 mL) to obtain a yellowish-brown solid, which was dried in vacuo. Yield: 12 mg, 42.2%. ¹H NMR (300 MHz, DMSO-d₆, ppm): δ 8.34 (s, 2H, NH₂), 8.01 (s, 1H, NH), 7.51 (d, J=9.0 Hz, 1H, ArH), 7.42 (d, J=9.0 Hz, 1H, ArH), 7.18 (s, 1H, C—H_{N-methylindole}), 7.04 (d, J=9.0 Hz, 1H, ArH), 6.63 (s, 6H, NH₃), 4.23 (s, 1H, CH), 4.07 (s, 2H, CH), 3.37 (s, 1H, CH), 3.24 (s, 2H, CH₂), 3.14 (s, 1H, CH), 2.85 (s, 2H, CH₂), 2.42 (s, 2H, CH₂), 2.27 (s, 2H, CH₂), 1.29 (s, 2H, CH₂(CH₂)₁₃CH₃), 1.20 (s, 26H, CH₂(CH₂)₁₃CH₃), 0.80 (t, 3H, CH₂(CH₂)₁₃CH₃). ¹³C NMR (125 MHz, DMSO-d₆, ppm): δ 175.0, 172.8, 170.3, 162.1, 140.3, 137.9, 130.4, 127.9, 122.5, 120.2, 119.6, 110.1, 108.0, 106.4, 100.4, 65.1, 53.7, 43.4, 40.7, 33.6, 32.4, 30.1, 27.2, 25.5, 22.6, 14.7. ¹⁹⁵Pt NMR (108 MHz, DMSO-d₆, ppm) δ 1248.0. HRMS (positive mode) C₃₅H₆₂Cl₂N₆O₇Pt: [M+H], calcd, (m/z) 945.3776; found 945.3769.

### Cell Lines and Cell Culture Conditions

Ovarian carcinoma cells, NIH:OVCAR3, A2780, A2780/CP70, and SKOV3 were purchased from the American Type Culture Collection (ATCC, Rockville Md.). Peripheral blood mononuclear cells (PBMCs) of two different donors were obtained from Zen Bio Inc. (Research Triangle, NC). Cancer cell lines were maintained in Roswell Park Memorial Institute (RPMI) medium (glutamine-free supplemented with 10% fetal bovine serum, Atlanta Biologics, Atlanta Ga.) and 1% penicillin/streptomycin. PBMCs were thawed in specialized medium (10% dextrose, 40% RPMI and 50% FBS) and subsequently cultured in RPMI containing 20% FBS. For T-cell proliferation experiments, PBMCs were stimulated by the addition of phytohemagglutinin (PHA-P). All cells were grown at 310 K in a humidified atmosphere containing 5% CO₂. D-1-methyltryptophan, L-tryptophan and L-kynurenine were purchased from Sigma Aldrich.

### RNAi Signatures

Compounds were added to achieve an LD₈₀₋₉₀ dose in Eμ-Mycp^{19arf-/-} cells by propidium iodide exclusion as determined by FACS after 48 h incubation. GFP enrichment/depletion was then determined by FACS at 72 h. Linkage ratios (LR) and p-values were generated as described previously.³⁻⁵ All FACS was conducted using a FACScan (BD Biosciences).

### GFP Competition Assays

Eμ-Mycp^{19arf-/-} lymphoma or p185⁺ BCR-Abl^{p19arf-/-} leukemia cells were infected with GFP-tagged shRNAs such that 15-25% of the population were GFP positive. An eighth of a million cells in 250 μL of B-cell media (BCM) were then seeded into 24-well plates. For wells that would remain untreated as a control, only 1/16th of a million cells were seeded. Next, 250 μL of media containing the compound of interest was added to the cells. After 24 h, 300 μL of cells from untreated wells were removed and replaced by 300 μL fresh BCM. All wells then received 500 μL BCM before being placed in the incubator for another 24 h. At 48 h, cells transduced with the control vector, MLS, were checked for viability via FACS on a FACScan (BD Biosciences) using propidium iodide as a live/dead marker. Untreated wells then had 700 μL of cells removed and replaced with 700 μL fresh media followed by a further 1 mL of fresh media. Wells that had compound sufficient to induce 80-90% cell kill (LD80-90) were then diluted by adding 1 mL of BCM. Finally, at 72 h, all wells for which an LD80-90 was achieved, as well as the untreated samples, were analysed via FACS to determine GFP % enrichment.

### Cellular Determination of Kynurenine and Tryptophan

HPLC analyses were performed using an Agilent HPLC with a diode array detector and Zorbax RP-18 column (250 mm×4 mm ID, 5 μm). SKOV3 cells were plated at 3×10⁵ cells in 2 mL medium containing 10% FBS, 100 U/mL penicillin, and 100 μg/mL streptomycin in a 6-well plate for kynurenine and tryptophan concentrations measurements. The medium was harvested after 48 h, centrifuged, and frozen until further analysis. After thawing, the samples were supplemented with 20% trichloroacetic acid for protein precipitation, centrifuged, and 100 μL of the supernatant was analyzed by HPLC. Standard curves were generated with kynurenine and tryptophan in the same medium. FBS contains kynurenine, owing to which low kynurenine concentrations (˜1 μM) were detected in all samples, and medium without cells was always measured for comparison.

### Quantitative (q)RT-PCR

Total RNA was isolated with the Qiagen RNAeasy RNA isolation kit (Hilden, Germany) and cDNA was synthesized with the Applied Biosystems reverse-transcription-kit (Foster City, Calif., USA). qRT-PCR was performed in a Light cycler 480 II thermo cycler with SYBR Green PCR Mastermix (Roche). All primers were separated by at least one intron on the genomic DNA to exclude its amplification. PCR reactions were checked by including no-RT controls, by omission of templates, and by melting curves. Standard curves were generated for each gene. Relative quantification of gene expression was determined by comparison of threshold values. All samples were analyzed in duplicate at two different dilutions. All results were normalized to GAPDH.

Primer sequences were (5′-3′ forward, reverse):
(SEQ ID NO: 1)
TGGCCAGCTTCGAGAAAGAG;
(IDO1)
(SEQ ID NO: 2)
TTGGCAAGACCTTACGGACA.
(SEQ ID NO: 3)
AATGGGCAGCCGTTAGGAAA;
(GAPDH)
(SEQ ID NO: 4)
GCCCAATACGACCAAATCAGAG.
(SEQ ID NO: 5)
ATTGTGCCGAGTCCCATATC;
(AHR)
(SEQ ID NO: 6)
AAGCAGGCGTGCATTAGACT.
(SEQ ID NO: 7)
AAATTCGGTACATCCTCGACGG;
(IL-6)
(SEQ ID NO: 8)
GGAAGGTTCAGGTTGTTTTCTGC.

### Western Blot Analysis

SKOV3 cells (1×10⁶ cells) were incubated with 2 (5 μM) for 24, 48, and 72 h at 37° C. Cells were washed with PBS, scraped into SDS-PAGE loading buffer (64 mM Tris-HCl (pH 6.8)/9.6% glycerol/2% SDS/5% β-mercaptoethanol/0.01% Bromophenol Blue), and incubated at 95° C. for 10 min. Whole cell lysates were resolved by 4-20% sodium dodecylsulphate polyacylamide gel electrophoresis (SDS-PAGE; 200 V for 25 min) followed by electro transfer to polyvinylidene difluoride membrane, PVDF (350 mA for 1 h). Membranes were blocked in 5% (w/v) bovine serum albumin in PBST (PBS/0.1% Tween 20) and incubated with the appropriate primary antibodies (Cell Signalling Technology and Santa Cruz). After incubation with horseradish peroxidase-conjugated secondary antibodies (Cell Signalling Technology), 1:1000, immuno complexes were detected with the ECL detection reagent (BioRad) and analyzed using UVP biospectrum imaging system fitted with a chemiluminescence filter.

### Cytotoxicity MTT Assay

The colorimetric MTT assay was used to determine the toxicity of 1, 2, and cisplatin. Cells (2×10³) were seeded in each well of a 96-well plate. After incubating the cells overnight, various concentrations of platinum as determined by graphite furnace atomic absorption spectroscopy (GF-AAS, Perkin-Elmer AAnalyst600) (0.03-50 μM) were added and incubated for 72 h (total volume 200 μL). Cisplatin was prepared as a 5 mM solution in PBS and diluted using media. 1, 2 and C₁₆—Pt-Suc-NHC₂H₄OH were prepared as 10 mM solution in DMSO and diluted using media. The final volume amount of DMSO in each well was 0.5%, and which was present in the untreated control as well. Cells co-incubated with cisplatin and (D)-1-MT were first pre-treated with 1 mM (D)-1-MT. After 72 h, the medium was removed, 200 μL of a 0.5 mg/mL solution of MTT in RPMI was added, and the plate was incubated for an additional 2-4 h. The RPMI/MTT, mixture was aspirated and 100 μL of DMSO was added to dissolve the resulting purple formazan crystals. The absorbance of the solution wells was read at 570 and 650 nm. Absorbance values were normalized to DMSO-containing control wells and plotted as concentration of test compound versus % cell viability. IC₅₀ values were interpolated from the resulting dose dependent curves. The reported IC₅₀ values are the average of at least three independent experiments, each of which consisted of six replicates per concentration level.

### Whole Cell Uptake Studies

1.5 million SKOV3 cells were seeded in 60 mm×10 mm petri dishes and incubated for 24 h at 37° C. Cells were then incubated with the test compounds (10 μM) in fresh medium (3 mL) and subsequently incubated for a given period of time at 37° C. The medium was then removed and cells were washed with PBS (3×1 mL), harvested by trypsinization (0.5 mL), and collected in a 1.5 mL eppendorf tube. The eppendorf tube containing the cell suspension was centrifuged at 2000 rpm for 5 min at 4° C., the resultant cell pellet was digested using 70% HNO₃, and the platinum content was analyzed by GFAAS to obtain the whole cell uptake.

### Intracellular Distribution

To measure cellular uptake, ca. 20 million SKOV3 cells were treated with 5 μM compounds 1, 2 and cisplatin at 37° C. for 17 h. The media were removed, and the cells were washed with PBS solution (1 mL×3), harvested, and centrifuged. The cell pellet was suspended in an appropriate volume of PBS to obtain a homogeneous suspension (100 μL). The nuclear and cytoplasmic extraction kit (NE-PER, Thermo Scientific Inc.) was used to extract the separate cytoplasmic, nuclear, and pelleted fractions. The fractions were mineralized with 70% HNO₃ and then heated at 95° C. for 10 min. The platinum content was analyzed by GF-AAS. Cellular platinum levels were expressed as pmol of Pt per million cells. Results are presented as the mean of three determinations for each data point.

### DNA Platination Experiments

SKOV3 (5×10⁶) cells were seeded in 100 mm×20 mm petri dishes and incubated at 37° C. until 80-90% confluent. These cells were then treated with the compounds (10 μM) in fresh medium (9 mL) and subsequently incubated for 17 h at 37° C. The medium was aspirated and the cells were washed with PBS (3×8 mL), harvested by trypsinization (1 mL), and washed with 2 mL of PBS. The cell suspension was centrifuged at 2000 rpm for 5 min at 4° C. The cell pellet was suspended in DNAzol (1 mL, genomic DNA isolation reagent,). The DNA was precipitated with pure ethanol (0.5 mL), washed with 75% ethanol (0.75 mL×3), and re-dissolved in 500 μL of 8 mM NaOH. The DNA concentration was determined by UV-Vis spectroscopy and the platinum content was quantified by GFAAS. All experiments were carried out in triplicate.

### Flow Cytometry Analysis

In order to monitor the cell cycle, flow cytometry studies were carried out. SKOV3 cells (5×10⁵) were seeded in 60 mm petri-dishes and incubated at 37° C. overnight. The cells were subsequently incubated with compound 1, 2 or cisplatin (5 μM) for 24, 48, and 72 h at 37° C. Cells were harvested from adherent cultures by trypsinization and combined with all detached cells from the incubation medium to assess total cell viability. Following centrifugation at 1000 rpm for 5 min, cells were washed with PBS and then fixed with 70% ethanol in PBS. Fixed cells were collected by centrifugation at 2500 rpm for 3 min, washed with PBS, and centrifuged as before. Cell pellets were treated with 100 μg/mL RNaseA (Qiagen) and re-suspended in 50 μg/mL propidium iodide (Sigma) in PBS for nucleic acid staining. The DNA content was measured on a FACSCalibur-HTS flow cytometer (BD Biosciences) using laser excitation at 488 nm and 20,000 events per sample were acquired. Cell cycle profiles were analyzed by using the ModFit software. For the apoptosis experiments, the Annexin V-SYTOX apoptosis-dead cells assay detection kit was used. The manufacture's protocol was followed to carry out this experiment. Briefly, untreated and treated cells (1×10⁵) were suspended in 1× annexin binding buffer (96 μL) (10 mM HEPES, 140 mM NaCl, 2.5 mM CaCl₂, pH 7.4), then 5 μL APC-annexin V and 2 μL SYTOX green (10 μg/mL) were added to each sample and the mixture was incubated on ice for 15 min. Subsequently, more binding buffer (150 μL) was added while gently mixing. The samples were kept on ice prior to being read on the FACS Calibur-HTS flow cytometer (BD Biosciences) and 20,000 events per sample were acquired. Cell populations were analyzed with the FlowJo software (Tree Star).

### Mixed Leukocyte Reaction (T-Cell Proliferation)

SKOV3 cells (2×10³) were seeded in 6-well plates. Cells were treated with compound 2 for 48 h and an untreated group was maintained as control. Peripheral blood mononuclear cells (PBMCs) from unrelated respondents were used. The T-cell population of PBMCs was measured by FACS Calibur-HTS flow cytometer (BD Biosciences) after staining with APC-anti CD3 antibody. PHA-P stimulated PBMCs (2×10⁵) and carboxyfluorescein succinimidyl ester (CFSE) stained PBMCs (2×10⁵) from unrelated donors were added in co-cultures of SKOV3 tumor cells (treated and untreated) and incubated for six days at 37° C., the and CFSE signal analyzed by flow cytometry on days 1 and 6.

### Whole Human Blood Stability Test

To a volume of 0.8 mL fresh whole human blood (Innovative Research, Novi, Mich.) was added 24 μL of a DMSO solution of 2 (2.0 mM), resulting in a final concentration of 60 μM. After 5 sec of vortexing, a 100 μL aliquot of the blood sample (0 h time point) was immediately transferred to a new tube containing 300 μL of octanol, and the remaining 500 μL of blood was incubated at 37° C. with rotation (1100 rpm). The blood-octanol mixture was vortexed for 2 min at R.T. and then centrifuged to separate the phases. The isolated octanol extract was diluted with methanol for GFAAS measurement. At varying time points (1 h, 2 h, 3 h, 5 h, and 7 h), a 100 μL aliquot was removed from the incubating blood and extracted as described above. For the 7 h sample, analytical HPLC was employed to confirm the integrity of the Pt compound. A standard of 2 and an octanol extract from blank whole human blood were used for comparison.

In Vivo Blood Stability:

Six weeks old female Balb/c mice were dosed with 8 mg/kg nanoparticle formulation of compound 2. Blood (200 μL) of mice were collected at varying time points (10 min, 1 h, 3 h, 6 h, and 24 h) and then extracted with octanol (300 μL). The octanol extracts were diluted with methanol and analysed by GFAAS.

### Nanoparticle Encapsulation of 2

The method for encapsulation followed an established protocol. Briefly, a 550 μL DMF solution containing 10 mg of PLGA-PEG-COOH and 2.5 mg of platinum complex 2 was prepared. A 500 μL aliquot of this solution was added in a dropwise manner over the course of 10 min to 5 mL of rapidly stirring Milli-Q water. The DMF solutions were added by a mechanical pipet, and nanoprecipitation was carried out in 20 mL glass scintillation vials. The water was stirred magnetically using a 0.5 cm stir bar at approximately 500 rpm. This suspension of nanoparticles was stirred for an additional 20 min and then passed through a 0.45 μm cellulose acetate syringe filter (VWR). The filtrate was loaded into an Amicon Centrifugal Filtration Device (100 kDa MWCO regenerated cellulose membrane). The loaded device was centrifuged at 1500 g for 20 min, concentrating the nanoparticle suspension to approximately 1 mL. This concentrated material was suspended in an additional 10 mL of fresh Milli-Q water and centrifuged again under identical conditions (3×). The final concentrated suspension was diluted to 1.5 mL with Milli-Q water for use in further experiments. The nanoparticle size was confirmed by dynamic light scattering.

### FPLC Experiments

To 2 mL of 400 μM HSA in PBS was added 0.4 mL of a DMSO solution of 2 (2 mM) under stirring at R.T. After 5 min, the clear solution was injected into a HiLoad 16/60 Superdex 200 size exclusion column equilibrated with PBS buffer. The absorbance of the effluent was detected at 280 nm. The flow rate was set to 1 mL/min and 3 mL fractions were collected. A control with only HSA was also carried out. The isolated fractions were further analyzed by GFAAS to determine their platinum content, and then stored at −40° C. for further use.

### Measurement of Water-Octanol Partition Coefficient (Log P)

The log P values for 1, 2 and (D)-1-MT were obtained by using the shake-flask method and GFAAS. Octanol used in this experiment was pre-saturated with PBS by overnight incubation with shaking of a biphasic mixture of the two at room temperature. A portion of 0.3 mL PBS containing 50 μM of 1 and 2 was incubated with the pre-saturated octanol (0.3 mL) in a 1.5 mL tube. The tube was shaken at room temperature for 3 h. The two phases were separated by centrifugation and the platinum content in each phase was determined by GFAAS. Owing to the low solubility of 2 in PBS, a pre-saturated octanol solution containing 50 μM of the platinum compound was used instead.

### Example 3

### Introduction

Indoleamine-2,3-dioxygenase (IDO) and tryptophan 2,3-dioxygenase (TDO) are two important enzymes that catalyze the rate limiting step in the catabolism of tryptophan (trp), the most energetic essential amino acid, to N-formyl-kynurenine, the first and rate limiting step of the kynurenine pathway.

TDO is restricted to the liver whereas IDO can be expressed in many cell types by proinflammatory cytokines such as interferon-γ (IFN-γ). Hence, IDO is expressed primarily in cells within the immune system, especially in dendritic cells and macrophages. Therefore, the overexpression of IDO has been implicated in a variety of diseases, including cancer.

IDO has been classified as an immune inhibitory checkpoint as experiments suggest that the depletion of trp also inhibits T cell proliferation. IDO has been shown to play a role in immune evasion by tumors since it mediates the degradation of trp suppressing T cell activation and inducing T cell apoptosis.

### IDO Inhibitors

The overexpression of IDO in tumor cells is considered to be a prognostic biomarker for reduced survival in cancer patients. Mechanistically, IDO is active with heme iron in the ferrous form (Fe²⁺) and inactive in ferric form (Fe³⁺). It is therefore inhibited when tryptophan binds to the ferric form.

Inhibitors have been shown to increase efficacy without increasing the toxicity of chemotherapeutic agents such as 1-methyltryptophan (1-MT), a weak competitive inhibitor (Kᵢ=34 μM).

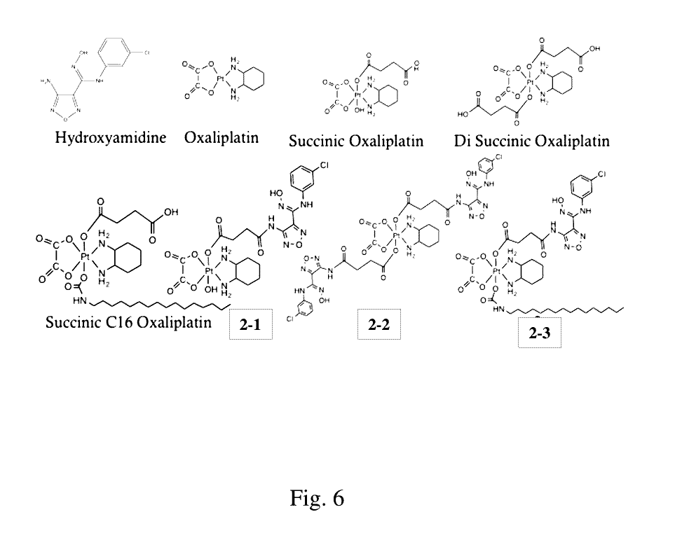

Other potent competitive inhibitors have been developed. Hydroxyamidine inhibitors, structure shown in FIG. 6, was investigated. Structure-activity relationships (SAR) show high potency for these compounds.

### Platinum Anticancer Complexes

It has been demonstrated in the 1960s that cisplatin inhibits cellular division of E. coli, and consequently as an effective antitumor agent. Cisplatin kills cancer cells by cross-linking DNA and inhibiting transcription. Cisplatin undergoes aquation when it enters the cell and loses one or both of its chloride ligands, creating an electrophile that can readily react. Purine bases in nucleic acid are highly nucleophilic at the N7 position, so cisplatin readily binds to it forming bifunctional adducts. The Pt-DNA adduct distorts and bends the DNA structure, impeding transcription ultimately leading to apoptosis.

However, although cisplatin was shown to be effective for testicular cancer, it is not for other cancer types and can cause toxic side effects. Many cancers are resistant to cisplatin therapy whether it was intrinsic or developed during prolonged treatment.

In this example, oxaliplatin was chosen to be investigated. Similar to cisplatin, it can induce DNA cross-linking. In addition, studies show that oxaliplatin can also induce immunogenic cell death (ICD). ICD is a specific pathway for cell death triggered by endoplasmic reticulum (ER) stress and the production of reactive oxygen species (ROS). This leads to the secretion of major damage associated molecular patterns (DAMPs), including calreticulin (CRT), HMGB1, and ATP. These DAMPs bind to immature dendritic cells, which grow to become antigen-presenting cells regulating effective T cell attacks. See FIG. 6 for compounds.

### Materials and Methods

### Synthesis

Hydroxyamidine IDO inhibitor was synthesized as known in the art (e.g., see Koblish, Holly K., Michael J. Hansbury, Kevin J. Bowman, Gengjie Yang, Claire L. Neilan, Patrick J. Haley, Timothy C. Burn, et al. “Hydroxyamidine Inhibitors of Indoleamine-2,3-Dioxygenase Potently Suppress Systemic Tryptophan Catabolism and the Growth of IDO-Expressing Tumors.” Molecular Cancer Therapeutics 9, no. 2 (Feb. 1, 2010): 489-98. doi:10.1158/1535-7163.MCT-09-0628). Hydroxyamidine required two intermediates following an established protocol.

Oxaliplatin was synthesized using known methods.

### Succinic Platinum (IV)

The oxidation of oxaliplatin from Pt(II) to Pt(IV) was carried for functionalizing the platinum complex to facilitate a coupling reaction with the hydroxyamidine IDO inhibitor and was carried out using known methods (e.g., see Zhang, J. Z., Bonnitcha, P., Wexselblatt, E., Klein, A. V., Najajreh, Y., Gibson, D., & Hambley, T. W. (2013). Facile Preparation of Mono-, Di- and Mixed-Carboxylato Platinum (IV) Complexes for Versatile Anticancer Prodrug Design. Chemistry-A European Journal, 19(5), 1672-1676). First a dihodroxy Platinum (IV) complex was synthesized (Oxa(IV)-2OH), followed by either a mono-succinic platinum (IV) complex (Oxa(IV)-Mono-COOH) or di-succinic platinum (IV) complex (Oxa(IV)-Di-COOH) following an established protocol.⁸ Scheme1 show the synthesis process.

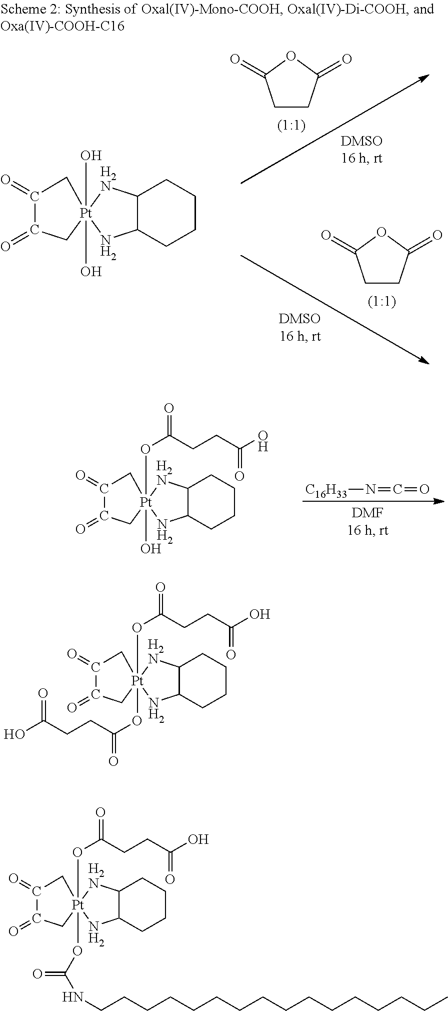

Platinum-Hydroxyamidine Conjugates:

A solution of hydroxyamidine (64 mg, 0.25 mmol) was prepared in DMF (4 mL). Oxa(IV)-Mono-COOH (100 mg, 0.2 mmol) and dimethylaminopyridine (DMAP) (6.5 mg, 0.05 mmol) was added to the solution, which was then cooled to 0° C. Dicyclohexylcarbodiimide (DCC) (154.75 mg, 0.75 mmol) was added and the mixture stirred at 0° C. for 5 mins. It was then allowed to warm to room temperature overnight. Precipitated urea (white solid) was filtered off and the remaining filtrate was lyophilized to remove the DMF. Dichloromethane (DCM) was used to wash off remaining urea. Due to difficulties, though, the final product was purified through HPLC. Oxa(IV)-COOH—C16 was used to synthesize the C16 version of the conjugate, and Oxa(IV)-Di-COOH with an increase of 4 fold of other reagents was used to synthesize the di-substituted version of the conjugate. (see Scheme 3).

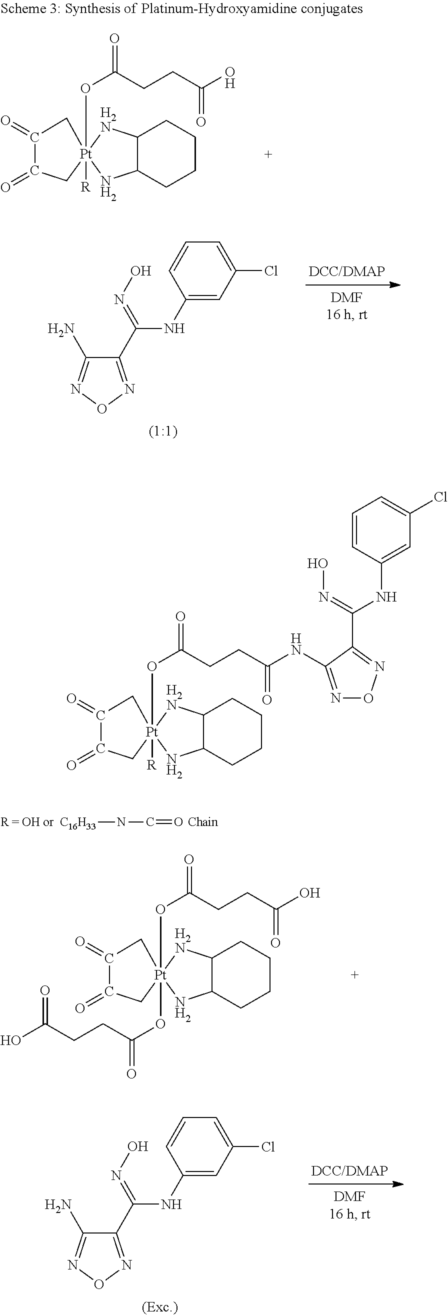

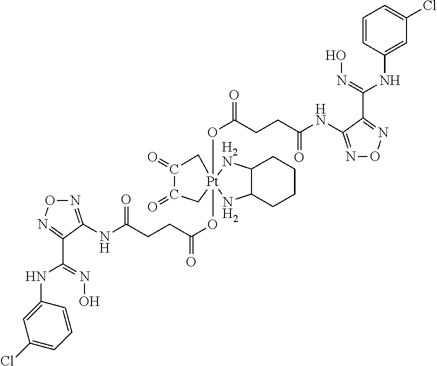

Purification of compounds 2-1, 2-2, and 2-3:

All three compounds underwent similar preliminary purification with organic washes using DMF, DCM, and acetonitrile to rid the mixture of the urea byproduct as well as precipitation in ether, which is commonly used with platinum complexes.

Compound 2-1 underwent purification using the preparative HPLC, which was not sufficient, so the semi preparative HPLC was used. Most recently, the combination of the biotage and semipreparative was used for purification, which yielded a purity of 97%.

Compound 2-1 underwent purification using column chromatography (90:10 ethyl acetate to ethanol solvent system) followed by semi preparative HPLC. Yielded 87% purity.

Compound 2-3 underwent purification using the preparative and semi preparative HPLC. Decomposition was observed.

### IDO Inhibition

A Western Blot and L-Kynurinine assay was performed to monitor and quantify IDO inhibition. SKOV3 Cells, expressive of the IDO enzyme, were plated in 2 mL RPMI media on a 6-Well plate. After 24 hours, with cell adhesion to the base, cells in three wells were treated with 5 μM Hydroxyamidine and the others were treated with 5 μM of compound 2-1. The media in all wells were spiked with 2.5 μM L-Tryptophan. After 48 hours, the media of each well was collected and frozen for the kynurenine assay while the cells were lysed, heated to 90° C. for 10 mins, and cooled then frozen for use in the western blot.

### Kynurenine Assay

The kynurenine assay was conducted by first measuring a calibration curve. Samples of 0.05, 0.5, 1, 1.5, 2.5, 5 μM of L-Tryptophan (Trp) and L-Kynurenine (Kyn) in water were run through the HPLC where UV absorption spectra of Trp (collected at 280 nm) and Kyn (collected 380 nm) were obtained and the peaks of interest were integrated for intensity values. These intensity values were then plotted against concentration (μM) to produce the calibration curve.

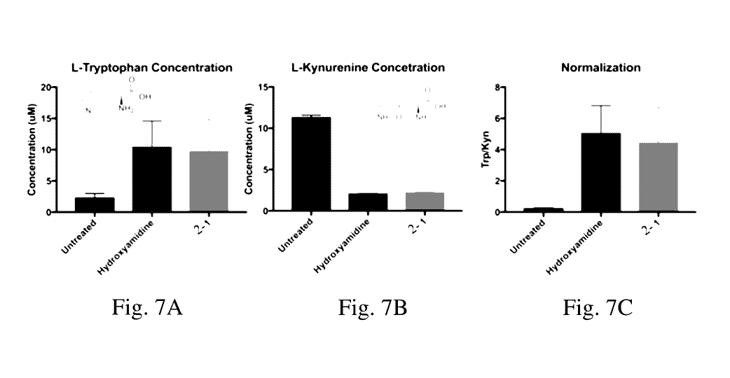

Afterwards, the media samples were run through the HPLC and the Trp and Kyn concentrations were calculated yielding FIG. 7A-FIG. 7C. This shows that Compound 2-1 reduces Trp/Kyn ratio levels by 22 fold, which is comparable to the hydroxyamidine control (25 fold).

### Western Blot

A blot was prepared by first thawing the cell lysates and preparing a 1 Liter 1×SDS solution. The gel apparatus was then assembled, filled with the SDS solution, and 14 μL of the lysate was injected in the wells in addition to 4 μL of the ladder. The voltage was then set to a constant 200 V for 30 minutes to run the gel. While running, the transfer buffer was prepared and placed in the cold room. When the gel is completed, 200 mL of methanol is added to 800 mL of transfer buffer, which was then used to soak the gel in the solution. A transfer membrane was cut to the same size as the gel and then soaked in methanol for 2 minutes and then soaked in the same solution as the gel.

The transfer apparatus was then assembled and placed in the transfer chamber. To begin transfer, the current was set constant at 350 mA for 60 minutes. When completed, the transfer membrane was placed in 2 mL blocking buffer and rocked on the shaker for 1 hour. β-Actin was chosen to be blotted as a control in addition to the IDO enzyme to monitor the loading of the gel. Therefore, one gel had 2 μL of rabbit IDO antibodies added to it while the other had mouse β-Actin antibodies instead. After the addition of the primary antibodies, the membranes were rocked overnight in the cold room.

The membranes were washed with PBS the next day, then a 3 time wash with PBST while rocking for 15 mins each time was commenced. When the washing cycles were complete, 2 mL of the blocking buffer and 2 μL of the secondary antibody (rabbit antibodies for the IDO and mouse antibodies for the β-Actin) was added and the membranes were incubated and rocked at room temperature for 45 minutes. The wash steps were then repeated and the gels were prepared for imaging.

The IDO band is observed for 2-1 and hydroxyamidine and therefore, IDO enzymatic activity is regulated through competitive binding inhibition rather than transcriptional degradation.

### Anticancer Activity

An MTT assay was run to measure the compounds anti cancer activity. The two colorectal cell lines HCT 116 P53+/+ and HCT 116 P53−/− were plated on 96 well plates. After adhesion to the plate, each individual compound was added to three rows, each consisting of 8 wells with a 3 fold dilution in concentration per well beginning at 25 μM. After 48 hours of incubation, the MTT reagent was added to the plates, which were then incubated for another 4 hours. The well contents were then dissolved in 200 μL of DMSO, and the plates were read for UV absorption at 570 nm and 650 nm. The collected data was then analyzed to produce the sigmoidal curves, which were then used to interpolate the IC50 values of each compounds. (See Table 3).

TABLE 3
IC50 values (μM) of all five tested compounds interpolated from toxicity
curves. Compound 2-3 shows to be most effective with the two cell lines.

IC50 Values (μM)

| | HCT116 | HCT116 |
|---|---|---|
| Comp. | p53 +/+ | p53 −/− |
| Hydroxyamidine | >100 | 54 ± 22.91 |
| Oxaliplatin | 1.322 ± 1.48 | 6.549 ± 1.78 |
| 2-1 | >100 | >100 |
| 2-2 | 18.64 ± 1.32 | 33.48 ± 6.46 |
| 2-3 | 1.479 ± 1.12 | 1.858 ± 2.81 |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A complex comprising Formula (I): 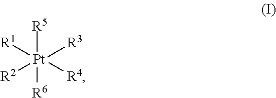
or a pharmaceutically acceptable salt thereof, wherein:
R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;
R⁵ and R⁶ can be the same or different and are —(Y)ₙR⁷, wherein each Y is the same or different and is selected from the group consisting of —O—, —NR⁸—, —C(═O)—, optionally substituted alkylene, optionally substituted heteroalkylene, optionally substituted arylene, and optionally substituted heteroarylene,
n is an integer between 1 and 10 inclusive,
each R⁷ is the same or different and is hydrogen, halo, optionally substituted alkyl, optionally substituted heteroalkyl, optionally substituted aryl, or optionally substituted heteroarylene, provided at least one R⁷ comprises an immune checkpoint inhibitor; and
each R⁸ is hydrogen, optionally substituted alkyl, or optionally substituted aryl.

2. The complex of claim 1, wherein:
R¹, R², R³, and R⁴ can be the same or different and each is a group comprising at least one of ammonia, an amine, an aryl group, a heterocycle including at least one nitrogen, or a leaving group, any being optionally substituted, or, any two or three of R¹, R², R³ and R⁴ can be joined together to form a bidentate ligand or tridentate ligand, any being optionally substituted;
R⁵ and R⁶ can be the same or different and are —(Y)ₙR⁷, wherein each Y is the same or different and is selected from the group consisting of —O—, —NR⁸—, —C(═O)—, optionally substituted alkylene, optionally substituted heteroalkylene, optionally substituted arylene, and optionally substituted heteroarylene;
n is an integer between 1 and 10 inclusive,
each R⁷ is the same or different and is hydrogen, halo, optionally substituted alkyl, optionally substituted heteroalkyl, optionally substituted aryl, or optionally substituted heteroarylene, provided at least one R⁷ comprises the structure: 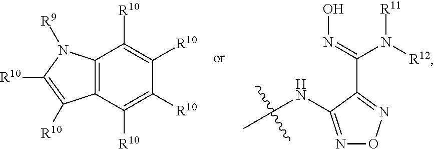
each R⁸ is hydrogen, optionally substituted alkyl, or optionally substituted aryl;
R⁹ is hydrogen, optionally substituted alkyl, or optionally substituted aryl;
each R¹⁰ is the same or different and is hydrogen, optionally substituted alkyl, optionally substituted heteroalkyl, optionally substituted aryl, or optionally substituted heteroaryl, provided at least one R¹⁰ is a bond to —(Y)ₙ—;
R¹¹ is hydrogen, optionally substituted alkyl, optionally substituted heteroalkyl, optionally substituted aryl, and optionally substituted heteroaryl; and
R¹² is optionally substituted aryl.

3. The complex of claim 2, wherein at least one or each of R⁵ and R⁶ comprises the structure:

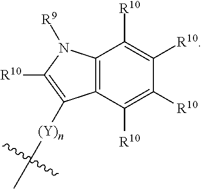

4. The complex of claim 2, wherein at least one or R⁵ and R⁶ comprises the structure or each of R⁵ and R⁶ comprises:

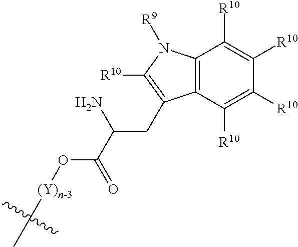

5. The complex of claim 2, wherein R⁵ comprises the structure:

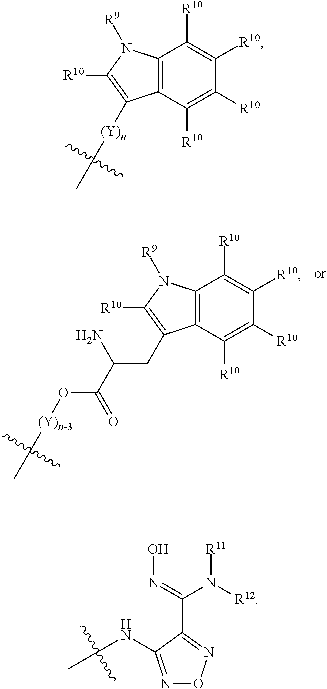

6. The complex of claim 4, wherein each R⁹ and R¹⁰ is hydrogen.

7. The complex of claim 5, wherein R¹¹ is hydrogen and R¹² is substituted phenyl.

8. The complex of claim 5, wherein —(Y)ₙ— is —O—C(═O)-alkylene-C(═O)—NH-alkylene-O—C(═O)—CHNH₂-alkylene-, —O—C(═O)—CHNH₂-alkylene- or —O—C(═O)-alkylene-(C═O)—.

9. The complex of claim 5, wherein R⁶ is —(Y)ₙ—(C₁₀₋₂₄ alkyl) or —O—C(═O)—NH—(C₁₀₋₂₄ alkyl).

10. The complex of claim 2, wherein each of R¹ and R² comprise the group NH₃ or R¹ and R² are linked and are —O(C═O)(C═O)O—.

11. The complex of claim 2, wherein each of R³ and R⁴ is chloro or R³ and R⁴ are linked and are diaminocyclohexane.

12. The complex of claim 2, wherein the dissociation of R⁵ and R⁶ from the platinum center forms a compound having Formula (II), 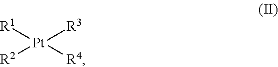
or a pharmaceutically acceptable salt thereof.

13. The complex of claim 12, wherein the compound having Formula (II) is a therapeutically active platinum(II) agent.

14. The complex of claim 12, wherein the compound having Formula (II) comprises cisplatin, carboplatin, or oxaliplatin, or a precursor thereof.

15. The complex of claim 12, wherein the compound having Formula (II) is active towards cancer.

15. The complex of claim 2, wherein the complex having Formula (I) has the formula 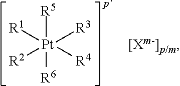
wherein:
X is a counterion;
p is 1 or 2; and
m is 1, 2, or 3.

16. The complex of claim 2, wherein the complex is selected from the group consisting of:

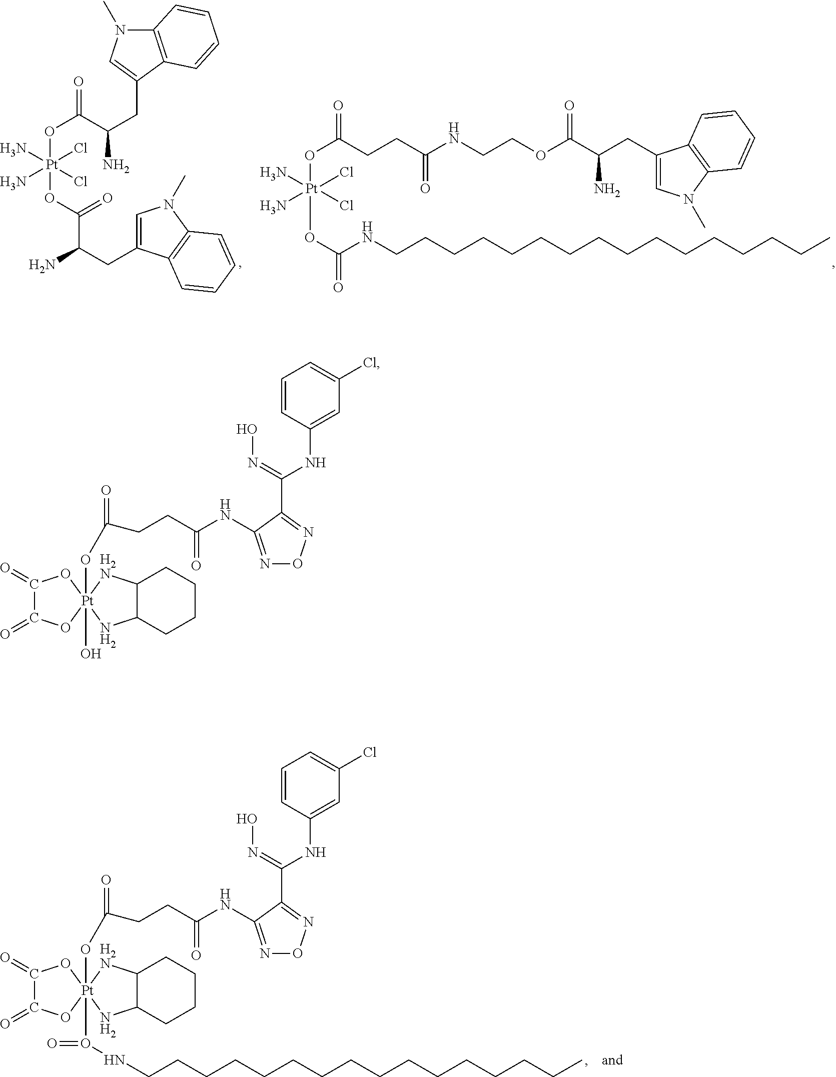

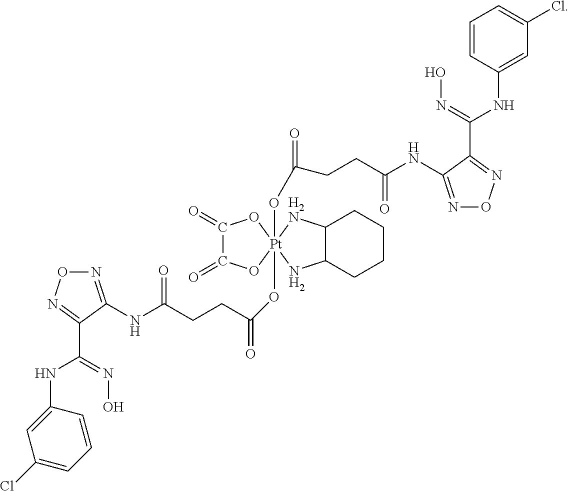

17. The complex of claim 2, wherein the complex or a pharmaceutically acceptable salt thereof is associated with and/or contained within a particle.

18. A pharmaceutical composition, comprising:
the complex of claim 2 or a pharmaceutically acceptable salt, thereof; and
one or more pharmaceutically acceptable carriers, additives and/or diluents.

19. A method of treating a patient in need of a therapeutic protocol, comprising:
administering to the patient the complex of claim 2 or a pharmaceutically acceptable salt thereof.


SEQUENCE LISTING (8 sequences)

SEQ ID NO: 1 - DNA, 20 bp, Artificial Sequence
  Synthetic Polynucleotide
tggccagcttcgagaaagag

SEQ ID NO: 2 - DNA, 20 bp, Artificial Sequence
  Synthetic Polynucleotide
ttggcaagaccttacggaca

SEQ ID NO: 3 - DNA, 20 bp, Artificial Sequence
  Synthetic Polynucleotide
aatgggcagccgttaggaaa

SEQ ID NO: 4 - DNA, 22 bp, Artificial Sequence
  Synthetic Polynucleotide
gcccaatacgaccaaatcagag

SEQ ID NO: 5 - DNA, 20 bp, Artificial Sequence
  Synthetic Polynucleotide
attgtgccgagtcccatatc

SEQ ID NO: 6 - DNA, 20 bp, Artificial Sequence
  Synthetic Polynucleotide
aagcaggcgtgcattagact

SEQ ID NO: 7 - DNA, 22 bp, Artificial Sequence
  Synthetic Polynucleotide
aaattcggtacatcctcgacgg

SEQ ID NO: 8 - DNA, 23 bp, Artificial Sequence
  Synthetic Polynucleotide
ggaaggttcaggttgttttctgc